SEQUENCE LISTING (100 sequences)

SEQ ID NO: 1 - DNA, 57 bp, Artificial Sequence
  Primer for plasmid constructiion as shown in Fig 18
ggaaaggtctcacagccgcnnnnnnncggagggtgcaagcgttaatcggaattactg

SEQ ID NO: 2 - DNA, 60 bp, Artificial Sequence
  Primer for plasmid construction
ggaaggtctcagctgcnnnnncggagttagccggtgcttcttctgcgggtaacgtcaatg

SEQ ID NO: 3 - DNA, 64 bp, Artificial Sequence
  Primer for plasmid construction
ggaaaggtctcacaccgcccnnnnnaccatgggagtgggttgcaaaagaagtaggtagcttaac

SEQ ID NO: 4 - DNA, 52 bp, Artificial Sequence
  Primer for plasmid construction
ggaaaggtctctggtgtgtacaaggcccgggaacgtattcaccgtggcattc

SEQ ID NO: 5 - DNA, 72 bp, Artificial Sequence
  Primer for plasmid construction
ggaaaggtctcactggggtnnnnnngtaacaaggtaaccgtaggggaacctgcggttggatcatgggattac

SEQ ID NO: 6 - DNA, 51 bp, Artificial Sequence
  Primer for plasmid construction
ggaaaggtctctccagtcatgaatcacaaagtggtaagcgccctcccgaag

SEQ ID NO: 7 - DNA, 62 bp, Artificial Sequence
  Primer for plasmid construction
ggaaaggtctcacttgtacnnnnnncccgtcacaccatgggagtgggttgcaaaagaagtag

SEQ ID NO: 8 - DNA, 49 bp, Artificial Sequence
  Primer for plasmid construction
ggaaaggtctctcaaggcccgggaacgtattcaccgtggcattctgatc

SEQ ID NO: 9 - DNA, 62 bp, Artificial Sequence
  Primer for plasmid construction
ggaaaggtctcagtcgtaannnnnnaaccgtaggggaacctgcggttggatcatgggattac

SEQ ID NO: 10 - DNA, 48 bp, Artificial Sequence
  Primer for plasmid construction
ggaaaggtctcacgacttcaccccagtcatgaatcacaaagtggtaag

SEQ ID NO: 11 - DNA, 57 bp, Artificial Sequence
  Primer for plasmid construction
ggaaaggtctcacaacgcgnngaaccttacctggtcttgacatccacggaagttttc

SEQ ID NO: 12 - DNA, 66 bp, Artificial Sequence
  Primer for plasmid construction
ggaaaggtctcagttgcatcgnnnnnnnncacatgctccaccgcttgtgcgggcccccgtcaattc

SEQ ID NO: 13 - DNA, 54 bp, Artificial Sequence
  Primer for plasmid construction
ggaaaggtctcaccagggctnnacacgtgctacaatggcgcatacaaagagaag

SEQ ID NO: 14 - DNA, 51 bp, Artificial Sequence
  Primer for plasmid construction
ggaaaggtctcactggtcgtaagggccatgatgacttgacgtcatccccac

SEQ ID NO: 15 - DNA, 73 bp, Artificial Sequence
  Primer for plasmid construction
ggaaaggtctctgtggtttaattnnnnnnnnnnnnaagaaccttacctggtcttgacatccacggaagttttc

SEQ ID NO: 16 - DNA, 50 bp, Artificial Sequence
  Primer for plasmid construction
ggaaaggtctcaccacatgctccaccgcttgtgcgggcccccgtcaattc

SEQ ID NO: 17 - DNA, 62 bp, Artificial Sequence
  Primer for plasmid construction
ggaaaggtctctcgtgagacagnnnnnnnntggctgtcgtcagctcgtgttgtgaaatgttg

SEQ ID NO: 18 - DNA, 43 bp, Artificial Sequence
  Primer for plasmid construction
ggaaaggtctctcacggttcccgaaggcacattctcatctctg

SEQ ID NO: 19 - DNA, 62 bp, Artificial Sequence
  Primer for plasmid construction
ggaaaggtctcacgtcaagtcatcannnnncttacgaccagggctacacacgtgctacaatg

SEQ ID NO: 20 - DNA, 44 bp, Artificial Sequence
  Primer for plasmid construction
ggaaaggtctctgacgtcatccccaccttcctccagtttatcac

SEQ ID NO: 21 - DNA, 63 bp, Artificial Sequence
  Primer for plasmid construction
ggaaaggtctcagatgacgnnnnnncatcatggcccttacgaccagggctacacacgtgctac

SEQ ID NO: 22 - DNA, 49 bp, Artificial Sequence
  Primer for plasmid construction
ggaaaggtctctcatccccaccttcctccagtttatcactggcagtctc

SEQ ID NO: 23 - DNA, 58 bp, Artificial Sequence
  Primer for plasmid construction
ggaaaggtctcactgcatgnnnnncgtcagctcgtgttgtgaaatgttgggttaagtc

SEQ ID NO: 24 - DNA, 47 bp, Artificial Sequence
  Primer for plasmid construction
ggaaaggtctcagcagcacctgtctcacggttcccgaaggcacattc

SEQ ID NO: 25 - DNA, 64 bp, Artificial Sequence
  Primer for plasmid construction
ggaaaggtctctgacgtcaagnnnnnatggcccttacgaccagggctacacacgtgctacaatg

SEQ ID NO: 26 - DNA, 52 bp, Artificial Sequence
  Primer for plasmid constructiion as shown in Fig 18
ggaaaggtctcacgtcatccccaccttcctccagtttatcactggcagtctc

SEQ ID NO: 27 - DNA, 58 bp, Artificial Sequence
  Primer for plasmid constructiion as shown in Fig 18
ggaaaggtctcactgcatnnctgtcgtcagctcgtgttgtgaaatgttgggttaagtc

SEQ ID NO: 28 - DNA, 47 bp, Artificial Sequence; identical to SEQ ID NO: 24
  Primer for plasmid constructiion as shown in Fig 18

SEQ ID NO: 29 - DNA, 68 bp, Artificial Sequence
  Primer for plasmid constructiion as shown in Fig 18
ggaaaggtctcagtggggatnnnnncaagtcatcatggcccttacgaccagggctacacacgtgctac

SEQ ID NO: 30 - DNA, 49 bp, Artificial Sequence
  Primer for plasmid constructiion as shown in Fig 18
ggaaaggtctctccaccttcctccagtttatcactggcagtctcctttg

SEQ ID NO: 31 - DNA, 64 bp, Artificial Sequence
  Primer for plasmid constructiion as shown in Fig 18
ggaaaggtctcaatggctgnnnnnagctcgtgttgtgaaatgttgggttaagtcccgcaacgag

SEQ ID NO: 32 - DNA, 51 bp, Artificial Sequence
  Primer for plasmid constructiion as shown in Fig 18
ggaaaggtctcaccatgcagcacctgtctcacggttcccgaaggcacattc

SEQ ID NO: 33 - DNA, 62 bp, Artificial Sequence
  Primer for plasmid constructiion as shown in Fig 18
ggaaaggtctcaggtgctnnnnnnctgtcgtcagctcgtgttgtgaaatgttgggttaagtc

SEQ ID NO: 34 - DNA, 45 bp, Artificial Sequence
  Primer for plasmid constructiion as shown in Fig 18
ggaaaggtctcacacctgtctcacggttcccgaaggcacattctc

SEQ ID NO: 35 - DNA, 64 bp, Artificial Sequence
  Primer for plasmid constructiion as shown in Fig 18
ggaaaggtctctgacgtcaagnnnnnnnggcccttacgaccagggctacacacgtgctacaatg

SEQ ID NO: 36 - DNA, 43 bp, Artificial Sequence
  Primer for plasmid constructiion as shown in Fig 18
ggaaaggtctcagcagcacctgtctcacggttcccgaaggcac

SEQ ID NO: 37 - DNA, 89 bp, Artificial Sequence
  Primer for plasmid constructiion as shown in Fig 18
ggaaaggtctctcagagatgagaatgtgccttcgggaaccgtgagacaggtgctgnatggctgtcgtcagctcgtgttgtgaaatgttg

SEQ ID NO: 38 - DNA, 95 bp, Artificial Sequence
  Primer for plasmid constructiion as shown in Fig 18
ggaaaggtctcatctgaaaacttccgtggatgtcaagaccaggtaaggttcttcgnntnncnncgaattaaaccacatgctccaccgcttgtgcg

SEQ ID NO: 39 - DNA, 67 bp, Artificial Sequence
  Primer for plasmid constructiion as shown in Fig 18
ggaaaggtctcagatgatgncnngtcatcatggcccttacgaccagggctacacacgtgctacaatg

SEQ ID NO: 40 - DNA, 54 bp, Artificial Sequence
  Primer for plasmid constructiion as shown in Fig 18
ggaaaggtctctcatccccaccttcctccagtttatcactggcagtctcctttg

SEQ ID NO: 41 - DNA, 36 bp, Artificial Sequence
  Primer for plasmid constructiion as shown in Fig 18
tgttcttcgtcaagagccaacccgtgggtcagcttc

SEQ ID NO: 42 - DNA, 36 bp, Artificial Sequence
  Primer for plasmid constructiion as shown in Fig 18
acgggttggctcttgacgaagaacatgttttcgatg

SEQ ID NO: 43 - DNA, 36 bp, Artificial Sequence
  Primer for plasmid constructiion as shown in Fig 18
tgttcttcgtcaggagccaacccgtgggtcagcttc

SEQ ID NO: 44 - DNA, 36 bp, Artificial Sequence
  Primer for plasmid constructiion as shown in Fig 18
acgggttggctcctgacgaagaacatgttttcgatg

SEQ ID NO: 45 - DNA, 37 bp, Artificial Sequence
  Primer for plasmid constructiion as shown in Fig 18
gaaaccttcaggaagcctgtggagcgaataccacgac

SEQ ID NO: 46 - DNA, 37 bp, Artificial Sequence
  Primer for plasmid constructiion as shown in Fig 18
cacaggcttcctgaaggtttcggtctgttcgtggaag

SEQ ID NO: 47 - DNA, 36 bp, Artificial Sequence
  Primer for plasmid constructiion as shown in Fig 18
tgttcttcgtcccctgccaacccgtgggtcagcttc

SEQ ID NO: 48 - DNA, 36 bp, Artificial Sequence
  Primer for plasmid constructiion as shown in Fig 18
acgggttggcaggggacgaagaacatgttttcgatg

SEQ ID NO: 49 - DNA, 37 bp, Artificial Sequence
  Primer for plasmid constructiion as shown in Fig 18
gaaaccttcccctagcctgtggagcgaataccacgac

SEQ ID NO: 50 - DNA, 37 bp, Artificial Sequence
  Primer for plasmid constructiion as shown in Fig 18
cacaggctaggggaaggtttcggtctgttcgtggaag

SEQ ID NO: 51 - DNA, 36 bp, Artificial Sequence
  Primer for plasmid constructiion as shown in Fig 18
tgttcttcgtctagagccaacccgtgggtcagcttc

SEQ ID NO: 52 - DNA, 36 bp, Artificial Sequence
  Primer for plasmid constructiion as shown in Fig 18
acgggttggctctagacgaagaacatgttttcgatg

SEQ ID NO: 53 - DNA, 37 bp, Artificial Sequence
  Primer for plasmid constructiion as shown in Fig 18
gaaaccttctagaagcctgtggagcgaataccacgac

SEQ ID NO: 54 - DNA, 37 bp, Artificial Sequence
  Primer for plasmid constructiion as shown in Fig 18
cacaggcttctagaaggtttcggtctgttcgtggaag

SEQ ID NO: 55 - DNA, 35 bp, Artificial Sequence
  Primer for plasmid constructiion as shown in Fig 18
accggtattcttacaccggagtaggggcaactcta

SEQ ID NO: 56 - DNA, 35 bp, Artificial Sequence
  Primer for plasmid constructiion as shown in Fig 18
ctccggtgtaagaataccggtccgttcagccgctc

SEQ ID NO: 57 - DNA, 35 bp, Artificial Sequence
  Primer for plasmid constructiion as shown in Fig 18
accggtcttcctaaaccggagtaggggcaactcta

SEQ ID NO: 58 - DNA, 35 bp, Artificial Sequence
  Primer for plasmid constructiion as shown in Fig 18
ctccggtttaggaagaccggtccgttcagccgctc

SEQ ID NO: 59 - DNA, 35 bp, Artificial Sequence
  Primer for plasmid constructiion as shown in Fig 18
accggtgtagggtaaccggagtaggggcaactcta

SEQ ID NO: 60 - DNA, 35 bp, Artificial Sequence
  Primer for plasmid constructiion as shown in Fig 18
ctccggttacccuacaccggtccgttcagccgctc

SEQ ID NO: 61 - DNA, 35 bp, Artificial Sequence
  Primer for plasmid constructiion as shown in Fig 18
accggtattctaacaccggagtaggggcaactcta

SEQ ID NO: 62 - DNA, 35 bp, Artificial Sequence
  Primer for plasmid constructiion as shown in Fig 18
ctccggtgtuagaataccggtccgttcagccgctc

SEQ ID NO: 63 - DNA, 63 bp, Artificial Sequence
  Primer for plasmid constructiion as shown in Fig 18
ggaaaggtctcagatgatgtcgggtcatcatggcccttacgaccagggctacacacgtgctac

SEQ ID NO: 64 - DNA, 63 bp, Artificial Sequence
  Primer for plasmid constructiion as shown in Fig 18
ggaaaggtctcagatgacgttgggtcatcatggcccttacgaccagggctacacacgtgctac

SEQ ID NO: 65 - DNA, 63 bp, Artificial Sequence
  Primer for plasmid constructiion as shown in Fig 18
ggaaaggtctcagatgacgtagagtcatcatggcccttacgaccagggctacacacgtgctac

SEQ ID NO: 66 - DNA, 49 bp, Artificial Sequence; identical to SEQ ID NO: 22
  Primer for plasmid constructiion as shown in Fig 18

SEQ ID NO: 67 - DNA, 37 bp, Artificial Sequence
  Primer for plasmid constructiion as shown in Fig 18
cgtgacgggaggactcaaaatcgaagaaggtaaactg

SEQ ID NO: 68 - DNA, 35 bp, Artificial Sequence
  Primer for plasmid constructiion as shown in Fig 18
tcttcgattttgagtcctcccgtcacgatgaattc

SEQ ID NO: 69 - DNA, 51 bp, Artificial Sequence
  Primer for plasmid constructiion as shown in Fig 18
gacgggaagactcaaaatcaggagaaggtaaactggtaatctggattaacg

SEQ ID NO: 70 - DNA, 50 bp, Artificial Sequence
  Primer for plasmid constructiion as shown in Fig 18
taccttctcttgattttgagtcctcccgtcacgatgaattcccggggatc

SEQ ID NO: 71 - DNA, 36 bp, Artificial Sequence
  Primer for plasmid constructiion as shown in Fig 18
cgataaaggcaggaaacggtctcgctgaagtccggt

SEQ ID NO: 72 - DNA, 35 bp, Artificial Sequence
  Primer for plasmid constructiion as shown in Fig 18
cgagaccgtttcctgcctttatcgccgttaatcca

SEQ ID NO: 73 - DNA, 35 bp, Artificial Sequence
  Primer for plasmid constructiion as shown in Fig 18
acaccgattactagatcgcagaagctgcctttaat

SEQ ID NO: 74 - DNA, 35 bp, Artificial Sequence
  Primer for plasmid constructiion as shown in Fig 18
gcttctgcgatctagtaatcggtgtctgcattcat

SEQ ID NO: 75 - DNA, 35 bp, Artificial Sequence
  Primer for plasmid constructiion as shown in Fig 18
gggcatggtcctagatcgacaccagcaaagtgaat

SEQ ID NO: 76 - DNA, 35 bp, Artificial Sequence
  Primer for plasmid constructiion as shown in Fig 18
ctggtgtcgatctaggaccatgcccacgggccgtt

SEQ ID NO: 77 - DNA, 35 bp, Artificial Sequence
  Primer for plasmid constructiion as shown in Fig 18
acgggaggactccaaatcgaatagggtaaactggt

SEQ ID NO: 78 - DNA, 35 bp, Artificial Sequence
  Primer for plasmid constructiion as shown in Fig 18
cctattcgatttggagtcctcccgtcacgatgaat

SEQ ID NO: 79 - DNA, 35 bp, Artificial Sequence
  Primer for plasmid constructiion as shown in Fig 18
cgaaaatcgaatagggtaaactggtaatctggatt

SEQ ID NO: 80 - DNA, 35 bp, Artificial Sequence
  Primer for plasmid constructiion as shown in Fig 18
accagtttaccctattcgattttcgatcctagagt

SEQ ID NO: 81 - DNA, 31 bp, Artificial Sequence
  Primer for plasmid constructiion as shown in Fig 18
cggcggacttcctaatccgcatgtcgctggt

SEQ ID NO: 82 - DNA, 31 bp, Artificial Sequence
  Primer for plasmid constructiion as shown in Fig 18
atgcggattaggaagtccgccgttctaccag

SEQ ID NO: 83 - DNA, 30 bp, Artificial Sequence
  Primer for plasmid constructiion as shown in Fig 18
aataccacaggagatttccggcagtttcta

SEQ ID NO: 84 - DNA, 30 bp, Artificial Sequence
  Primer for plasmid constructiion as shown in Fig 18
cggaaatctcctgtggtattcactccagag

SEQ ID NO: 85 - DNA, 35 bp, Artificial Sequence
  Primer for plasmid constructiion as shown in Fig 18
aaatcgaaaggaggtaaactggtaatctggattaa

SEQ ID NO: 86 - DNA, 35 bp, Artificial Sequence
  Primer for plasmid constructiion as shown in Fig 18
agtttacctcctttcgattttgagctacccgtcac

SEQ ID NO: 87 - DNA, 35 bp, Artificial Sequence
  Primer for plasmid constructiion as shown in Fig 18
tggttctgaggagaaggtgaatggcagctggttct

SEQ ID NO: 88 - DNA, 35 bp, Artificial Sequence
  Primer for plasmid constructiion as shown in Fig 18
tcaccttctcctcagaaccatggttaattcctcct

SEQ ID NO: 89 - DNA, 29 bp, Artificial Sequence
  Primer for plasmid constructiion as shown in Fig 18
ccaggatcctcgggagttgtcagcctgtc

SEQ ID NO: 90 - DNA, 28 bp, Artificial Sequence
  Primer for plasmid constructiion as shown in Fig 18
atggtcgaccgccgaacgcggcgttttg

SEQ ID NO: 91 - DNA, 30 bp, Artificial Sequence
  Primer for plasmid constructiion as shown in Fig 18
gcggtcgacacagatgtaggtgttccacag

SEQ ID NO: 92 - DNA, 31 bp, Artificial Sequence
  Primer for plasmid constructiion as shown in Fig 18
tatgcggccgccagaacatatccatcgcgtc

SEQ ID NO: 93 - DNA, 32 bp, Artificial Sequence
  Primer for plasmid constructiion as shown in Fig 18
cgggaattcaagctgaccaactgacagaagag

SEQ ID NO: 94 - DNA, 49 bp, Artificial Sequence
  Primer for plasmid constructiion as shown in Fig 18
actaagcttagtgatggtgatggtgatgctttgctgtcatcatttgtac

SEQ ID NO: 95 - DNA, 35 bp, Artificial Sequence
  Primer for plasmid constructiion as shown in Fig 18
gcgtggatccaggagctgaccaactgacagaagag

SEQ ID NO: 96 - DNA, 35 bp, Artificial Sequence
  Primer for plasmid constructiion as shown in Fig 18
gttggtcagctcctggatccacgcggaaccagatc

SEQ ID NO: 97 - DNA, 35 bp, Artificial Sequence
  Primer for plasmid constructiion as shown in Fig 18
tgatgacagcatagcatcaccatcaccatcactaa

SEQ ID NO: 98 - DNA, 35 bp, Artificial Sequence
  Primer for plasmid constructiion as shown in Fig 18
atggtgatgctatgctgtcatcatttgtacaaact

SEQ ID NO: 99 - DNA, 35 bp, Artificial Sequence
  Primer for plasmid constructiion as shown in Fig 18
tgaggtcgctttagcaaaacccaacggaagcagaa

SEQ ID NO: 100 - DNA, 35 bp, Artificial Sequence
  Primer for plasmid constructiion as shown in Fig 18
gttgggttttgctaaagcgacctcataacggtgcc

(19) United States
(12) Patent Application Publication
(10) Pub. No.: US 2012/0264926 A1
(43) Pub. Date: Oct. 18, 2012

(54) Orthogonal Q-Ribosomes

(75) Inventors: Jason Chin, Cambridge (GB); Kaihang Wang, Cambridge (GB); Heinz Neumann, Gottingen (DE)

(73) Assignee: MEDICAL RESEARCH COUNCIL, Swindon (GB)

(21) Appl. No.: 13/517,372

(22) PCT Filed: Dec. 20, 2010
(86) PCT No.: PCT/GB2010/002296

§ 371 Date: Jun. 20, 2012

Related U.S. Application Data
(60) Provisional application No. 61/288,421, filed on Dec. 21, 2009.

(30) Foreign Application Priority Data
Dec. 21, 2009 (GB) 0922351.2

(51) Int. Cl. C07H 21/02; C07H 1/00

(52) U.S. Cl. 536/23.1; 536/25.3

(57) ABSTRACT

The invention relates to 16S rRNA comprising a mutation at A1196, and to 16S rRNA further comprising a mutation at C1195 and/or A1197, and to 16S rRNA which comprises (i) C1195A and A1196G; or (ii) C1195T, A1196G and A1197G; or (iii) A1196G and A1197G. The invention also relates to ribosomes comprising such 16S rRNAs and to use of same.

### FIELD OF THE INVENTION

The invention relates to ribosomes for translation of quadruplet codons.

### BACKGROUND TO THE INVENTION

Since each of the 64 triplet codons are used to encode natural amino acids or polypeptide termination, new blank codons are required for cellular genetic code expansion. In principle quadruplet codons might provide 256 blank codons.

Stoichiometrically aminoacylated extended anticodon tRNAs have been used to incorporate unnatural amino acids in response to 4-base codons with very low efficiency in in vitro systems¹¹⁻¹³ and in limited in vivo systems, via import of previously aminoacylated tRNA^{14 15}. This is a problem in the art.

In one case a 4-base suppressor and amber codon have been used, in a non-generalizable approach, to encode two unremarkable amino acids with low efficiency¹⁶. Indeed, the inefficiency with which natural ribosomes decode quadruplet codons severely limits their utility for genetic code expansion, which is a problem in the art.

The present invention seeks to overcome problem(s) associated with the prior art.

### SUMMARY OF THE INVENTION

The inventors have mutated certain ribosomal components to produce a ribosome with a new technical capability of translating quadruplet codons. The mutations have focussed on the 16S rRNA. The ribosomes produced according to the present invention are sometimes referred to as quadruplet-ribosomes or Q-Ribosomes (RiboQ).

In one aspect, the invention relates to a 16S rRNA comprising a mutation at A1196.

In one aspect, the invention relates to a 16S rRNA comprising a mutation at A1196 and at least one further mutation selected from C1195T, A1197G, C1195A.

In another aspect, the invention relates to a 16S rRNA as described above further comprising a mutation at C1195 and/or A1197.

In another aspect, the invention relates to a 16S rRNA as described above which comprises

(i) C1195A and A1196G; or

(ii) C1195T, A1196G and A1197G; or

(iii) A1196G and A1197G.

In another aspect, the invention relates to a ribosome capable of translating a quadruplet codon, said ribosome comprising a 16S rRNA as described above.

In another aspect, the invention relates to use of a 16S rRNA as described above in the translation of a mRNA comprising at least one quadruplet codon.

### DETAILED DESCRIPTION OF THE INVENTION

In one aspect the invention relates to a 16S rRNA comprising a mutation at A1196.

Suitably said mutation is A1196G.

In another aspect, the invention relates to a 16S rRNA as described above further comprising a mutation at C1195 and/or A1197.

In another aspect, the invention relates to a 16S rRNA as described above which comprises

(i) C1195A and A1196G; or

(ii) C1195T, A1196G and A1197G; or

(iii) A1196G and A1197G.

In another aspect, the invention relates to a 16S rRNA as described above which further comprises A531 G and U534A.

In another aspect, the invention relates to a ribosome capable of translating a quadruplet codon, said ribosome comprising a 16S rRNA as described above.

In another aspect, the invention relates to use of a 16S rRNA as described above in the translation of a mRNA comprising at least one quadruplet codon.

Suitably the 16S rRNA of the invention comprising a mutation at A1196 comprises A1196G. This specific mutation is common to each of the preferred 16S rRNAs exemplified herein such as Q1, Q2, Q3 and Q4, which all possess A1196G (i.e. G at position 1196).

Suitably the 16S rRNA of the invention further comprises a mutation at A1197. Suitably the 16S rRNA of the invention comprising a mutation at A1197 comprises A1197G. This specific mutation is common to 75% of the preferred 16S rRNAs exemplified herein such as Q1, Q2 and Q3, which all possess A1197G (i.e. G at position 1197).

Suitably the 16S rRNA of the invention comprises a mutation at A1196 and a mutation at A1197. Most suitably the 16S rRNA of the invention comprises A1196G and A1197G. Each of Q1, Q2 and Q3 comprise this combination of mutations.

Suitably the 16S rRNA of the invention may comprise a mutation at C1195. This mutation may be C1195T or C1195A. Suitably the 16S rRNA of the invention which comprises a C1195 mutation also comprises a A1196 mutation such as A1196G. Suitably when the 16S rRNA of the invention comprises A1197G, it also comprises C1195T. Suitably when the 16S rRNA of the invention comprises A1196G and A1197G, it also comprises C1195T. Suitably when the 16S rRNA of the invention comprises A11 96G and is wild type at A1197 (i.e. A at position 1197), it also comprises C1195A.

Further mutations may be present or may not be present.

### Ribo-X and Ribo-Q

The Ribo-Q 16S rRNA sequences herein have been prepared from Ribo-X as a starting 16S rRNA sequence. Ribo-X is a published 16S rRNA sequence well known to the person skilled in the art. More specifically, Ribo-X refers to a 16S rRNA sequence which has two substitutions compared to wild type, namely A531 G and U534A. Therefore suitably each Ribo-Q 16S rRNA sequence described herein also possesses A531G and U534A in addition to each further mutation or substitution discussed herein. It should be assumed that the 16S rRNAs of the invention each possess A531 G and U534A in addition to any other mutations discussed, unless the context indicates otherwise. Thus, suitably each 16S rRNA of the invention comprises at least 3 mutations compared to wild type, namely A1196, A531G and U534A, most suitably A1196G, A531G and U534A.

In case any more detail is needed, Ribo-X is discussed in depth in PCT/GB2007/004562 (published as WO2008/065398). This document is specifically incorporated herein by reference expressly for the detail of the Ribo-X 16S rRNA sequence which is the ‘background’ or parent sequence from which the Ribo-Q 16S rRNAs of the invention are derived and/or produced.

Suitably the 16S rRNA of the invention comprises A1196G and A1197G (Ribo-Q1, Ribo-Q2, Ribo-Q3).

Suitably the 16S rRNA of the invention comprises C1195T and A1196G and A1197G (Ribo-Q3).

Suitably the 16S rRNA of the invention comprises C1195T and A1196G (Ribo-Q4).

In one embodiment the 16S rRNA of the invention consists of wild type 16S rRNA sequence and A531G and U534A and A1196G and A1197G (Ribo-Q1).

In one embodiment the 16S rRNA of the invention consists of wild type 16S rRNA sequence and A531G and U534A and A1196G and A1197G and up to 8 further mutations/substitutions (Ribo-Q2).

In one embodiment the 16S rRNA of the invention consists of wild type 16S rRNA sequence and A531G and U534A and C1195T and A1196G and A1197G (Ribo-Q3).

In one embodiment the 16S rRNA of the invention consists of wild type 16S rRNA sequence and A531G and U534A and C1195T and A1196G (Ribo-Q4).

The invention relates to encoding multiple unnatural amino acids via evolution of a quadruplet decoding ribosome.

### Definitions

As the term “orthogonal” is used herein, it refers to a nucleic acid, for example rRNA or mRNA, which differs from natural, endogenous nucleic acid in its ability to cooperate with other nucleic acids. Orthogonal mRNA, rRNA and tRNA are provided in matched groups (cognate groups) which cooperate efficiently. For example, orthogonal rRNA, when part of a ribosome, will efficiently translate matched cognate orthogonal mRNA, but not natural, endogenous mRNA. For simplicity, a ribosome comprising an orthogonal rRNA is referred to herein as an “orthogonal ribosome,” and an orthogonal ribosome will efficiently translate a cognate orthogonal mRNA.

An orthogonal codon or orthogonal mRNA codon is a codon in orthogonal mRNA which is only translated by a cognate orthogonal ribosome, or translated more efficiently, or differently, by a cognate orthogonal ribosome than by a natural, endogenous ribosome. Orthogonal is abbreviated to O (as in O-mRNA).

Thus, by way of example, orthogonal ribosome (O-ribosome)•orthogonal mRNA (O-mRNA) pairs are composed of: an mRNA containing a ribosome binding site that does not direct translation by the endogenous ribosome, and an orthogonal ribosome that efficiently and specifically translates the orthogonal mRNA, but does not appreciably translate cellular mRNAs.

“Evolved”, as applied herein for example in the expression “evolved orthogonal ribosome”, refers to the development of a function of a molecule through diversification and selection. For example, a library of rRNA molecules diversified at desired positions can be subjected to selection according to the procedures described herein. An evolved rRNA is obtained by the selection process.

As used herein, the term “mRNA” when used in the context of an O-mRNA O-ribosome pair refers to an mRNA that comprises an orthogonal codon which is efficiently translated by a cognate O-ribosome, but not by a natural, wild-type ribosome. In addition, it may comprise an mutant ribosome binding site (particularly the sequence from the AUG initiation codon upstream to −13 relative to the AUG) that efficiently mediates the initiation of translation by the O-ribosome, but not by a wild-type ribosome. The remainder of the mRNA can vary, such that placing the coding sequence for any protein downstream of that ribosome binding site will result in an mRNA that is translated efficiently by the orthogonal ribosome, but not by an endogenous ribosome.

As used herein, the term “rRNA” when used in the context of an O-mRNA O-ribosome pair refers to a rRNA mutated such that the rRNA is an orthogonal rRNA, and a ribosome containing it is an orthogonal ribosome, i.e., it efficiently translates only a cognate orthogonal mRNA. The primary, secondary and tertiary structures of wild-type ribosomal rRNAs are very well known, as are the functions of the various conserved structures (stems-loops, hairpins, hinges, etc.). O-rRNA typically comprises a mutation in 16S rRNA which is responsible for binding of tRNA during the translation process. It may also comprise mutations in the 3′ regions of the small rRNA subunit which are responsible for the initiation of translation and interaction with the ribosome binding site of mRNA.

The expression of an “O-rRNA” in a cell, as the term is used herein, is not toxic to the cell. Toxicity is measured by cell death, or alternatively, by a slowing in the growth rate by 80% or more relative to a cell that does not express the “O-mRNA.” Expression of an O-rRNA will preferably slow growth by less than 50%, preferably less than 25%, more preferably less than 10%, and more preferably still, not at all, relative to the growth of similar cells lacking the O-rRNA.

As used herein, the terms “more efficiently translates” and “more efficiently mediates translation” mean that a given O-mRNA is translated by a cognate O-ribosome at least 25% more efficiently, and preferably at least 2, 3, 4 or 8 or more times as efficiently as an O-mRNA is translated by a wild-type ribosome or a non-cognate O-ribosome in the same cell or cell type. As a gauge, for example, one may evaluate translation efficiency relative to the translation of an O-mRNA encoding chloramphenicol acetyl transferase using at least one orthogonal codon by a natural or non-cognate orthogonal ribosome.

As used herein, the term “corresponding to” when used in reference to nucleotide sequence means that a given sequence in one molecule, e.g., in a 16S rRNA, is in the same position in another molecule, e.g., a 16S rRNA from another species. By “in the same position” is meant that the “corresponding” sequences are aligned with each other when aligned using the BLAST sequence alignment algorithm “BLAST 2 Sequences” described by Tatusova and Madden (1999, “Blast 2 sequences—a new tool for comparing protein and nucleotide sequences”, FEMS Microbiol. Lett. 174:247-250) and available from the U.S. National Center for Biotechnology Information (NCBI). To avoid any doubt, the BLAST version 2.2.11 (available for use on the NCBI website or, alternatively, available for download from that site) is used, with default parameters as follows: program, blastn; reward for a match, 1; penalty for a mismatch, −2; open gap and extend gap penalties 5 and 2, respectively; gap×dropoff, 50; expect 10.0; word size 11; and filter on.

As used herein, the term “selectable marker” refers to a gene sequence that permits selection for cells in a population that encode and express that gene sequence by the addition of a corresponding selection agent.

As used herein, the term “region comprising sequence that interacts with mRNA at the ribosome binding site” refers to a region of sequence comprising the nucleotides near the 3′ terminus of 16S rRNA that physically interact, e.g., by base pairing or other interaction, with mRNA during the initiation of translation. The “region” includes nucleotides that base pair or otherwise physically interact with nucleotides in mRNA at the ribosome binding site, and nucleotides within five nucleotides 5′ or 3′ of such nucleotides. Also included in this “region” are bases corresponding to nucleotides 722 and 723 of the E. coli 16S rRNA, which form a bulge proximal to the minor groove of the Shine-Delgarno helix formed between the ribosome and mRNA.

As used herein, the term “diversified” means that individual members of a library will vary in sequence at a given site. Methods of introducing diversity are well known to those skilled in the art, and can introduce random or less than fully random diversity at a given site. By “fully random” is meant that a given nucleotide can be any of G, A, T, or C (or in RNA, any of G, A, U and C). By “less than fully random” is meant that a given site can be occupied by more than one different nucleotide, but not all of G, A, T (U in RNA) or C, for example where diversity permits either G or A, but not U or C, or permits G, A, or U but not C at a given site.

As used herein, the term “ribosome binding site” refers to the region of an mRNA that is bound by the ribosome at the initiation of translation. As defined herein, the “ribosome binding site” of prokaryotic mRNAs includes the Shine-Delgarno consensus sequence and nucleotides −13 to +1 relative to the AUG initiation codon.

As used herein, the term “unnatural amino acid” refers to an amino acid other than the 20 amino acids that occur naturally in protein. Non-limiting examples include: a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl) alanine, a 3-methyl-phenylalanine, an O-4-allyl-L-tyrosine, a 4-propyl-L-tyrosine, a tri-O-acetyl-GIcNAcb-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an a, a disubstituted amino acid; a b-amino acid; a cyclic amino acid other than proline or histidine, and an aromatic amino acid other than phenylalanine, tyrosine or tryptophan.

International patent application PCT/GB2006/002637 describes the generation of orthogonal ribosome/mRNA pairs in which the ribosome binding site in the O-mRNA binds specifically to the O-ribosome.

Briefly, the bacterial ribosome is a 2.5 MDa complex of rRNA and protein responsible for translation of mRNA into protein (The Ribosome, Vol. LXVI. (Cold Spring Harbor Laboratory Press, Cold Spring Harbor, N.Y.; 2001). The interaction between the mRNA and the 30S subunit of the ribosome is an early event in translation (Laursen, B. S., Sorensen, H. P., Mortensen, K. K. & Sperling-Petersen, H. U., Microbiol Mol Biol Rev 69, 101-123 (2005)), and several features of the mRNA are known to control the expression of a gene, including the first codon (Wikstrom, P. M., Lind, L. K., Berg, D. E. & Bjork, G. R., J Mol Biol 224, 949-966 (1992)), the ribosome-binding sequence (including the Shine Delgarno (SD) sequence (Shine, J. & Delgarno, L., Biochem J 141, 609-615 (1974), Steitz, J. A. & Jakes, K., Proc Natl Acad Sci U S A 72, 4734-4738 (1975), Yusupova, G. Z., Yusupov, M. M., Cate, J. H. & Noller, H. F., Cell 106, 233-241 (2001)), and the spacing between these sequences (Chen, H., Bjerknes, M., Kumar, R. & Jay, E., Nucleic Acids Res 22, 4953-4957 (1994)). In certain cases mRNA structure (Gottesman, S. et al. in The Ribosome, Vol. LXVI (Cold Spring Harbor Laboratory Press, Cold Spring Harbor, N.Y.; 2001), Looman, A. C., Bodlaender, J., de Gruyter, M., Vogelaar, A. & van Knippenberg, P. H., Nucleic Acids Res 14, 5481-5497 (1986)), Liebhaber, S. A., Cash, F. & Eshleman, S. S., J Mol Biol 226, 609-621 (1992), or metabolite binding (Winkler, W., Nahvi, A. & Breaker, R. R., Nature 419, 952-956 (2002)), influences translation initiation, and in rare cases mRNAs can be translated without a SD sequence, though translation of these sequences is inefficient (Laursen, B. S., Sorensen, H. P., Mortensen, K. K. & Sperling-Petersen, H. U., Microbiol Mol Biol Rev 69, 101-123 (2005)), and operates through an alternate initiation pathway, Laursen, B.S., Sorensen, H. P., Mortensen, K. K. & Sperling-Petersen, H. U. Initiation of protein synthesis in bacteria. Microbial Mol Biol Rev 69, 101-123 (2005). For the vast majority of bacterial genes the SD region of the mRNA is a major determinant of translational efficiency. The classic SD sequence GGAGG interacts through RNA-RNA base-pairing with a region at the 3′ end of the 16S rRNA containing the sequence CCUCC, known as the Anti Shine Delgarno (ASD). In E. coli there are an estimated 4,122 translational starts (Shultzaberger, R. K., Bucheimer, R. E., Rudd, K. E. & Schneider, TD., J Mol Biol 313, 215-228 (2001)), and these differ in the spacing between the SD-like sequence and the AUG start codon, the degree of complementarity between the SD-like sequence and the ribosome, and the exact region of sequence at the 3′ end of the 16S rRNA with which the mRNA interacts. The ribosome therefore drives translation from a more complex set of sequences than just the classic Shine Delgarno (SD) sequence. For clarity, mRNA sequences believed to bind the 3′ end of 16S rRNA are referred to as SD sequences and to the specific sequence GGAGG is referred to as the classic SD sequence.

Mutations in the SD sequence often lead to rapid cell lysis and death (Lee, K., Holland-Staley, C. A. & Cunningham, P. R., RNA 2, 1270-1285 (1996), Wood, T. K. & Peretti, S. W., Biotechnol. Bioeng 38, 891-906 (1991)). Such mutant ribosomes mis-regulate cellular translation and are not orthogonal. The sensitivity of cell survival to mutations in the ASD region is underscored by the observation that even a single change in the ASD can lead to cell death through catastrophic and global mis-regulation of proteome synthesis (Jacob, W. F., Santer, M. & Dahlberg, A. E., Proc Natl Acad Sci U S A 84, 4757-4761 (1987). Other mutations in the rRNA can lead to inadequacies in processing or assembly of functional ribosomes.

PCT/GB2006/02637 describes methods for tailoring the molecular specificity of duplicated E. coli ribosome mRNA pairs with respect to the wild-type ribosome and mRNAs to produce multiple orthogonal ribosome orthogonal mRNA pairs. In these pairs the ribosome efficiently translates only the orthogonal mRNA and the orthogonal mRNA is not an efficient substrate for cellular ribosomes. Orthogonal ribosomes as described therein that do not translate endogenous mRNAs permit specific translation of desired cognate mRNAs without interfering with cellular gene expression. The network of interactions between these orthogonal pairs is predicted and measured, and it is shown that orthogonal ribosome mRNA pairs can be used to post-transcriptionally program the cell with Boolean logic.

PCT/GB2006/02637 describes a mechanism for positive and negative selection for evolution of orthogonal translational machinery. The selection methods are applied to evolving multiple orthogonal ribosome mRNA pairs (O-ribosome O-mRNA). Also described is the successful prediction of the network of interactions between cognate and non-cognate O-ribosomes and O-mRNAs.

Here we provide new, further modified orthogonal ribosomes and methods for producing such O-ribosomes which expand the molecular decoding properties of the ribosome. Specifically, we evolve orthogonal ribosomes that more efficiently decode quadruplet codons.

We disclose evolved orthogonal ribosomes which enhance the efficiency of synthetic genetic code expansion. We provide cellular modules composed of an orthogonal ribosome and an orthogonal mRNA. These pairs function in parallel with, but independent of, the natural ribosome-mRNA pair in Escherichia coli. Orthogonal ribosomes do not synthesize the proteome and may be diverged to operate using different tRNA decoding rules from natural ribosomes. Here we demonstrate the evolution of orthogonal ribosomes (ribo-Q's) for the efficient, high fidelity decoding of codons such as quadruplet codons placed within the context of an orthogonal mRNA in living cells. We combine ribo-Q, orthogonal mRNAs and orthogonal aminoacyl-tRNA synthetase/tRNA pairs to substantially increase the efficiency of site-specific unnatural amino acid incorporation in E. coli. This advantageously allows the efficient synthesis of proteins incorporating unnatural amino acids at multiple sites, and/or minimizes the functional and/or phenotypic effects of truncated proteins for example in experiments that use unnatural amino acid incorporation to probe protein function in vivo.

### Orthogonal Codons

We describe an evolved ribosome which is capable of translating an orthogonal mRNA codon, which means that the ribosome interprets mRNA information according to a code which is not the universal genetic code, but an orthogonal genetic code. This introduces a number of possibilities, including the possibility of having two separate genetic systems present in the cell, wherein cross-talk is eliminated by virtue of the difference in code; or of a mRNA molecule encoding different polypeptides according to which code is used to translate it.

An orthogonal codon, from which orthogonal genetic codes can be assembled, is a code which is other than the universal triplet code. Table 1 below represents the universal genetic code:

TABLE 1
Second nucleotide

| | U | C | A | G | |
|---|---|---|---|---|---|
| U | UUU Phenylalanine (Phe) | UCU Serine (Ser) | UAU Tyrosine (Tyr) | UGU Cysteine | U |
| | | | | (Cys) | |
| | UUC Phe | UCC Ser | UAC Tyr | UGC Cys | C |
| | UUA Leucine (Leu) | UCA Ser | UAA STOP | UGA STOP | A |
| | UUG Leu | UCG Ser | UAG STOP | UGG Tryptophan | G |
| | | | | (Trp) | |
| C | CUU Leucine (Leu) | CCU Proline | CAU Histidine (His) | CGU Arginine | U |
| | | (Pro) | | (Arg) | |
| | CUC Leu | CCC Pro | CAC His | CGC Arg | C |
| | CUA Leu | CCA Pro | CAA Glutamine | CGA Arg | A |
| | | | (Gln) | | |
| | CUG Leu | CCG Pro | CAG Gln | CGG Arg | G |
| A | AUU Isoleucine (Ile) | ACU Threonine | AAU Asparagine | AGU Serine (Ser) | U |
| | | (Thr) | (Asn) | | |
| | AUC Ile | ACC Thr | AAC Asn | AGC Ser | C |
| | AUA Ile | ACA Thr | AAA Lysine (Lys) | AGA Arginine | A |
| | | | | (Arg) | |
| | AUG Methionine (Met) | ACG Thr | AAG Lys | AGG Arg | G |
| | or START | | | | |
| G | GUU Valine Val | GCU Alanine | GAU Aspartic acid | GGU Glycine | U |
| | | (Ala) | (Asp) | (Gly) | |
| | GUC (Val) | GCC Ala | GAC Asp | GGC Gly | C |
| | GUA Val | GCA Ala | GAA Glutamic acid | GGA Gly | A |
| | | | (Glu) | | |
| | GUG Val | GCG Ala | GAG Glu | GGG Gly | G |

Certain variations in this code occur naturally; for example, mitochondria use UGA to encode tryptophan (Trp) rather than as a chain terminator. In addition, most animal mitochondria use AUA for methionine not isoleucine and all vertebrate mitochondria use AGA and AGG as chain terminators.

Yeast mitochondria assign all codons beginning with CU to threonine instead of leucine (which is still encoded by UUA and UUG as it is in cytosolic mRNA).

Plant mitochondria use the universal code, and this has permitted angiosperms to transfer mitochondrial genes to their nucleus with great ease.

Violations of the universal code are far rarer for nuclear genes. A few unicellular eukaryotes have been found that use one or two (of their three) STOP codons for amino acids instead.

The vast majority of proteins are assembled from the 20 amino acids listed above even though some of these may be chemically altered, e.g. by phosphorylation, at a later time.

However, two cases have been found in nature where an amino acid that is not one of the standard 20 is inserted by a tRNA into the growing polypeptide.

Selenocysteine. This amino acid is encoded by UGA. UGA is still used as a chain terminator, but the translation machinery is able to discriminate when a UGA codon should be used for selenocysteine rather than STOP. This codon usage has been found in certain Archaea, eubacteria, and animals (humans synthesize 25 different proteins containing selenium).

Pyrrolysine. In one gene found in a member of the Archaea, this amino acid is encoded by UAG, How the translation machinery knows when it encounters UAG whether to insert a tRNA with pyrrolysine or to stop translation is not yet known.

All of the above are, for the purposes of the present invention, considered to be part of the universal genetic code.

The present invention enables novel codes, not previously known in nature, to be developed and used in the context of orthogonal mRNA/rRNA pairs.

### Selection for Orthogonal Ribosomes

A selection approach for the identification of orthogonal ribosome orthogonal mRNA pairs, or other pairs of orthogonal molecules, requires selection for translation of orthogonal codons in O-mRNA. The selection is advantageously positive selection, such that cells which express O-mRNA are selected over those that do not, or do so less efficiently.

A number of different positive selection agents can be used. The most common selection strategies involve conditional survival on antibiotics. Of these positive selections, the chloramphenicol acetyl-transferase gene in combination with the antibiotic chloramphenicol has proved one of the most useful. Others as known in the art, such as ampicillin, kanamycin, tetracycline or streptomycin resistance, among others, can also be used.

O-mRNA/O-rRNA pairs can be used to produce an orthogonal transcript in a host cell, for example CAT, that can only be translated by the cognate orthogonal ribosome, thereby permitting extremely sensitive control of the expression of a polypeptide encoded by the transcript. The pairs can thus be used to produce a polypeptide of interest by, for example, introducing nucleic acid encoding such a pair to a cell, where the orthogonal mRNA encodes the polypeptide of interest. The translation of the orthogonal mRNA by the orthogonal ribosome results in production of the polypeptide of interest. It is contemplated that polypeptides produced in cells encoding orthogonal mRNA•orthogonal ribosome pairs can include unnatural amino acids.

The methods described herein are applicable to the selection of orthogonal mRNA orthogonal rRNA pairs in species in which the O-mRNA comprises orthogonal codons which are translated by the O-rRNA. Thus, the methods are broadly applicable across prokaryotic and eukaryotic species, in which this mechanism is conserved. The sequence of 16S rRNA is known for a large number of bacterial species and has itself been used to generate phylogenetic trees defining the evolutionary relationships between the bacterial species (reviewed, for example, by Ludwig & Schleifer, 1994, FEMS Microbiol. Rev. 15: 155-73; see also Bergey's Manual of Systematic Bacteriology Volumes 1 and 2, Springer, George M. Garrity, ed.). The Ribosomal Database Project II (Cole J R, Chai B, Farris R J, Wang Q, Kulam S A, McGarrell D M, Garrity G M, Tiedje J M, Nucleic Acids Res, (2005) 33(Database Issue):D294-D296. doi: 10.1093/nar/gki038) provides, in release 9.28 (Jun. 17, 2005), 155,708 aligned and annotated 16S rRNA sequences, along with online analysis tools.

Phylogenetic trees are constructed using, for example, 16S rRNA sequences and the neighbour joining method in the ClustalW sequence alignment algorithm. Using a phylogenetic tree, one can approximate the likelihood that a given set of mutations (on 16S rRNA and a codon in mRNA) that render the set orthogonal with respect to each other in one species will have a similar effect in another species. Thus, the mutations rendering mRNA/16S rRNA pairs orthogonal with respect to each other in one member of, for example, the Enterobacteriaceae Family (e.g., E. coli) would be more likely to result in orthogonal mRNA/orthogonal ribosome pairs in another member of the same Family (e.g., Salmonella) than in a member of a different Family on the phylogenetic tree.

In some instances, where bacterial species are very closely related, it may be possible to introduce corresponding 16S rRNA and mRNA mutations that result in orthogonal molecules in one species into the closely related species to generate an orthogonal mRNA orthogonal rRNA pair in the related species. Also where bacterial species very are closely related (e.g., for E. coli and Salmonella species), it may be possible to introduce orthogonal 16S rRNA and orthogonal mRNA from one species directly to the closely related species to obtain a functional orthogonal mRNA orthogonal ribosome pair in the related species.

Alternatively, where the species in which one wishes to identify orthogonal mRNA orthogonal ribosome pairs is not closely related (e.g., where they are not in the same phylogenetic Family) to a species in which a set of pairs has already been selected, one can use selection methods as described herein to generate orthogonal mRNA orthogonal ribosome pairs in the desired species. Briefly, one can prepare a library of mutated orthogonal 16S rRNA molecules. The library can then be introduced to the chosen species. One or more O-mRNA sequences can be generated which comprise a sequence encoding a selection polypeptide as described herein using one or more orthogonal codons (the bacterial species must be sensitive to the activity of the selection agents, a matter easily determined by one of skill in the art). The O-mRNA library can then be introduced to cells comprising the O-rRNA library, followed by positive selection for those cells expressing the positive selectable marker in order to identify orthogonal ribosomes that pair with the O-mRNA.

The methods described herein are applicable to the identification of molecules useful to direct translation or other processes in a wide range of bacteria, including bacteria of industrial and agricultural importance as well as pathogenic bacteria. Pathogenic bacteria are well known to those of skill in the art, and sequence information, including not only 16S rRNA sequence, but also numerous mRNA coding sequences, are available in public databases, such as GenBank. Common, but non-limiting examples include, e.g., Salmonella species, Clostridium species, e.g., Clostridium botulinum and Clostridium perfringens, Staphylococcus sp., e.g, Staphylococcus aureus; Campylobacter species, e.g., Campylobacter jejuni, Yersinia species, e.g., Yersinia pestis, Yersinia enterocolitica and Yersinia pseudotuberculosis, Listeria species, e.g., Listeria monocytogenes, Vibrio species, e.g., Vibrio cholerae, Vibrio parahaemolyticus and Vibrio vulnificus, Bacillus cereus, Aeromonas species, e.g., Aeromonas hydrophila, Shigella species, Streptococcus species, e.g., Streptococcus pyogenes, Streptococcus faecalis, Streptococcus faecium, Streptococcus pneumoniae, Streptococcus durans, and Streptococcus avium, Mycobacterium tuberculosis, Klebsiella species, Enterobacter species, Proteus species, Citrobacter species, Aerobacter species, Providencia species, Neisseria species, e.g., Neisseria gonorrhea and Neisseria meningitidis, Heamophilus species, e.g., Haemophilus influenzae, Helicobacter species, e.g., Helicobacter pylori, Bordetella species, e.g., Bordetella pertussis, Serratia species, and pathogenic species of E. coli, e.g., Enterotoxigenic E. coli (ETEC), enteropathogenic E. coli (EPEC) and enterohemorrhagic E. coli O157:H7 (EHEC).

### Release Factor 1/Amber Codons

Advantageously, to maximize the efficiency of full-length protein synthesis with respect to truncated protein, the effects of release factor 1 (RF-1)-mediated chain termination would be minimized for the expression of a gene of interest.

Unlike the natural ribosome the orthogonal ribosome is not responsible for synthesizing the proteome, and is therefore tolerant to mutations in the highly conserved rRNA that cause lethal or dominant negative effects in the natural ribosome. Orthogonal ribosomes may therefore be advantageously evolved towards decreased RF-1 binding.

We disclose the synthetic evolution of orthogonal ribosomes (ribo-Q's) for the efficient, high fidelity decoding of quadruplet codons placed within the context of an orthogonal mRNA in living cells. Ribo-Q's may preferably be combined with orthogonal mRNAs and orthogonal aminoacyl-tRNA synthetase/tRNA pairs to advantageously significantly increase the efficiency of site-specific unnatural amino acid incorporation in E. coli. This increase in efficiency makes it possible to synthesize proteins incorporating unnatural amino acids at multiple sites, and minimizes the functional and phenotypic effects of truncated proteins in vivo. This has clear industrial application and utility, for example in the manufacture of proteins incorporating unnatural amino acids.

### Bacterial Transformation

The methods described herein rely upon the introduction of foreign or exogenous nucleic acids into bacteria. Methods for bacterial transformation with exogenous nucleic acid, and particularly for rendering cells competent to take up exogenous nucleic acid, is well known in the art. For example, Gram negative bacteria such as E. coli are rendered transformation competent by treatment with multivalent cationic agents such as calcium chloride or rubidium chloride. Gram positive bacteria can be incubated with degradative enzymes to remove the peptidoglycan layer and thus form protoplasts. When the protoplasts are incubated with DNA and polyethylene glycol, one obtains cell fusion and concomitant DNA uptake. In both of these examples, if the DNA is linear, it tends to be sensitive to nucleases so that transformation is most efficient when it involves the use of covalently closed circular DNA. Alternatively, nuclease-deficient cells (RecBC-strains) can be used to improve transformation.

Electroporation is also well known for the introduction of nucleic acid to bacterial cells. Methods are well known, for example, for electroporation of Gram negative bacteria such as E. coli, but are also well known for the electroporation of Gram positive bacteria, such as Enterococcus faecalis, among others, as described, e.g., by Dunny et al., 1991, Appl. Environ. Microbiol. 57: 1194-1201.

The in vivo, genetically programmed incorporation of designer amino acids allows the properties of proteins to be tailored with molecular precision¹. The Methanococcus jannaschii tyrosyl-tRNA synthetase/tRNA_{CUA} (MjT\dot{y}rRS/tRNA_{CUA})^{2, 3} and the Methanosarcina barkeri pyrrolysyl-tRNA synthetase/tRNA_{CUA} (MbPyIRS/tRNA_{CUA})⁴⁻⁶ orthogonal pairs have been evolved to incorporate a range of unnatural amino acids in response to the amber codon in E. coli^{1, 6, 7}. However, the potential of synthetic genetic code expansion is generally limited to the low efficiency incorporation of a single type of unnatural amino acid at a time, since every triplet codon in the universal genetic code is used in encoding the synthesis of the proteome. In order to efficiently encode multiple distinct unnatural amino acid into proteins we require i) blank codons and ii) mutually orthogonal aminoacyl-tRNA synthetase/tRNA pairs that recognize unnatural amino acids and decode the new codons. Here we synthetically evolve an orthogonal ribosome^{8, 9} (riboQ1) that efficiently decodes a series of quadruplet codons and the amber codon, providing several blank codons on an orthogonal mRNA, which it specifically translates⁸. By creating mutually orthogonal aminoacyl-tRNA synthetase/tRNA pairs and combining these with riboQ1 we direct the incorporation of distinct unnatural amino acids in response to two of the new blank codons on the orthogonal mRNA (FIG. 5). Using this code, we genetically direct the formation of a specific, redox insensitive, nanoscale protein cross-link via the bio-orthogonal cycloaddition of encoded azide and alkyne containing amino acids¹⁰. Since the synthetase/tRNA pairs used have been evolved to incorporate numerous unnatural amino acids^{1, 6, 7} it will be possible to encode more than 200 unnatural amino acid combinations using this approach. Since ribo-Q1 independently decodes a series of quadruplet codons this work provides foundational technologies for the encoded synthesis and synthetic evolution of unnatural polymers in cells.

A ribosome must accommodate an extended anticodon tRNA into its decoding centre to decode it^{17, 18}. Natural ribosomes are very inefficient at, and unevolvable for quadruplet decoding (FIG. 6), which would enhance misreading of the proteome. In contrast orthogonal ribosomes⁸, which are specifically addressed to the orthogonal message, and are not responsible for synthesizing the proteome, may, in principle, be evolved to efficiently decode quadruplet codons on the orthogonal message. To discover evolved orthogonal ribosomes that enhance quadruplet decoding we first created 11 saturation mutagenesis libraries in the 16S rRNA of ribo-X (an orthogonal ribosome previously evolved for efficient amber codon decoding on an orthogonal message⁹; taken together these libraries cover 127 nucleotides that are within 12 Å of a tRNA bound in the decoding centre¹⁹ (FIG. 7). We used ribo-X as a starting point for library generation because we hoped to discover evolved orthogonal ribosomes that gain the ability to efficiently decode quadruplet codons while maintaining the ability to efficiently decode amber codons on the orthogonal mRNA; thereby maximizing the number of additional codons that can be decoded on the orthogonal ribosome.

To select orthogonal ribosomes that efficiently decode quadruplet codons using extended anticodon tRNAs we combined each O-ribosome library with a reporter construct (O-cat (AAGA 146)/tRNA^{Ser2}_{UCUU}). The reporter contains a chloramphenicol acetyl transferase gene that is specifically translated by O-ribosomes⁹, an in frame AAGA quadruplet codon and tRNA^{Ser2}_{UCUU} (a designed variant of tRNA^{Ser2} that is aminoacylated by E. coli seryl-tRNA synthetase and decodes the AAGA codon^{9, 20}). The orthogonal cat gene is read in frame, and confers chloramphenicol resistance, only if tRNA^{Ser2}_{UCUU} efficiently decodes the AAGA codon and restores the reading frame. Clones surviving on chloramphenicol concentrations which kill cells containing ribo-X and the cat reporter have 4 distinct sequences. Clone ribo-Q4 has double mutations at C1195A and A1196G, ribo-Q3 has the triple mutations at C1195T, A1196G and A1197G; ribo-Q2 and ribo-Q1 have the double mutation at A1196G and A1197G, ribo-Q2 also has eight additional non-programmed mutations. While the entire decoding centre was mutated, the selected mutations are spatially localized and might accommodate an extended anticodon:codon interaction in the decoding centre (FIG. 1a). The chloramphenicol resistance of cells containing tRNA^{ser2}_{UCUU} and cat with two AGGA codons is greatly enhanced when the cat gene is translated by ribo-Q ribosomes in place of unevolved ribosomes (FIG. 1b,c). Indeed the chloramphenicol resistance of cells containing two AGGA codons read by the riboQ ribosomes approaches that of a wild-type cat gene. This suggests that riboQl may decode quadruplet codons with an efficiency approaching that for triplet decoding and with a much greater efficiency than the unevolved ribosome. The enhancement in quadruplet decoding efficiency is maintained for a variety of quadruplet codon-anticodon interactions (FIG. 8).

Natural ribosomes decode triplet codons with high fidelity (error frequencies ranging from 10⁻² to 10⁻⁴ errors per codon have been reported²¹⁻²³). To explicitly compare the fidelity of triplet decoding and quadruplet decoding for the evolved orthogonal ribosomes and the progenitor ribosome we used two independent methods: the incorporation of ³⁵S cysteine into a protein, which contains no cysteine codons in its gene⁹ and variants of a dual luciferase systems^{9, 23} (FIG. 9). We find that the triplet and quadruplet decoding translational fidelity is the same for the evolved ribosome (ribo-Q1) and un-evolved and wild-type ribosomes, and that the 4th base of the codon-anticodon interaction is discriminated equally well by all ribosomes (FIG. 9).

To demonstrate that the enhanced amber decoding properties of ribo-X are maintained in ribo-Q1 we compared the efficiency of incorporating p-benzoyl-L-phenylalanine (Bpa, 1) into a recombinant GST-MBP fusion in response to an amber codon on an orthogonal mRNA using orthogonal ribosomes and a previously evolved p-benzoyl-L-phenylalanyl-tRNA synthetase/tRNA_{CUA} pair³ (BpaRS/RNA_{CUA}) (FIG. 2). Ribo-Q1 and ribo-X incorporate 1 with a comparable and high efficiency in response to the amber codons in the orthogonal mRNA (compare lanes 4 & 6 and lanes 10 & 12 in FIG. 2a). Ribo-X and ribo-Q1 are substantially more efficient than the wild type ribosome at incorporating 1 via amber suppression (compare lanes 4 & 6 to lane 2 & lanes 10 & 12 to lane 8 in FIG. 2a).

To demonstrate the utility of ribo-Q1 for incorporating unnatural amino acids in response to quadruplet codons we compared the efficiency of incorporating p-azido-L-phenylalanine (AzPhe, 2) into a recombinant GST-MBP fusion in response to a quadruplet codon using ribo-Q1 or the wild-type ribosome. In order to direct the incorporation of 2 we used the AzPheRS*/tRNA_{UCCU} pair (a variant of the pAzPheRS-7/tRNA_{CUA} pair²⁴ derived from the MjTyrRS/tRNA_{CUA} pair for the incorporation of 2 as described below). We find that ribo-Q1 substantially increases the efficiency of incorporation of 2 in response to a quadruplet codon, and even allows the incorporation of 2 in response to two quadruplet codons for the first time (compare lanes 2 & 6 and lanes 4 & 8, FIG. 2b). The site and fidelity of incorporation of 2 were further confirmed by analysis of tandem mass spectrometry (MS/MS) fragmentation series of the relevant tryptic peptides (FIG. 11).

To take advantage of ribo-Q1 for the incorporation of multiple distinct unnatural amino acids in recombinant proteins, we required mutually orthogonal aminoacyl-tRNA synthetase/tRNA pairs. We demonstrated that the MbPyIRS/tRNA_{CUA} pair^{4, 5} and MjTyrRS/tRNA_{CUA} pair², each of which have previously been evolved to incorporate a range of unnatural amino acids^{1, 6, 7, 25}, are mutually orthogonal in their aminoacylation specificity (FIG. 12). We created the AzPheRS*/tRNA_{UCCU} pair, which is derived from the MjTyrRS/tRNA_{CUA} pair, by a series of generally applicable directed evolution steps (FIGS. 13-15). The MbPyIRS/tRNA_{CUA} pair and AzPheRS*/tRNA_{UCCU} pair are mutually orthogonal: they decode distinct codons, use distinct amino acids and are orthogonal in their aminoacylation specificity (FIG. 16).

To demonstrate the simultaneous incorporation of two useful unnatural amino acids into a single protein we combined the MbPyIRS/MbtRNA_{CUA} pair, the AzPheRS* tRNA_{UCCU} pair and ribo-Q1 in E. coli. We used these components to produce full-length GST-calmodulin containing 2 (AzPh_{.}e) and N6-[(2-propynyloxy)carbonyl]-L-lysine (CAK, 4, which we recently discovered is an efficient substrate for MbPyIRS⁷) (FIG. 3) in response to an AGGA and UAG codon in an orthogonal gene. Production of the full-length protein required the addition of both unnatural amino acids. We further confirmed the incorporation of 2 and 4 at the genetically programmed sites by MS/MS sequencing of a single tryptic fragment containing both unnatural amino acids (FIG. 3).

To begin to demonstrate that emergent properties may be programmed into proteins via combinations of unnatural amino acids we genetically directed the formation of a triazole cross-link, via a copper catalysed Husigen [2+3] cycloaddition reaction (“Click reaction”).¹⁰ We first encoded 2 and 4 at position 1 and 149 in calmodulin (FIG. 4). After incubation of calmodulin incorporating the azide (2) and alkyne (4) at these positions with Cu (I) for 5 minutes we observe a more rapidly migrating protein band in SDS-PAGE. MS/MS sequencing unambiguously confirms that the faster mobility band results from the product of bio-orthogonal cycloaddition reaction between 2 and 4. Our results demonstrate the genetically programmed proximity acceleration of a new class of asymmetric, redox insensitive cross-link that can be used to specifically constrain protein structure on the nanometer scale. Unlike existing protein cyclization methods for recombinant proteins^{26, 27}, these cross-links can be encoded at any spatially compatible sites in a protein, not just placed at the termini. In contrast to the chemically diverse cyclization methods that can be accessed with peptides by solid-phase peptide synthesis²⁸ these cross-links can be encoded into proteins of essentially any size. Given the importance of disulfide bonds in natural therapeutic proteins and hormones, the utility of peptide stapling strategies²⁹, the importance of peptide cyclization³⁰, and the improved stability of proteins cyclized by native chemical ligation²⁶ it will be interesting to investigate the enhancement of protein function that may be accessed by combining the encoding of these cross-links with directed evolution methods. By combining the numerous variant MjTyrRS/tRNA_{CUA} and MbPyIRS/tRNA_{CUA} pairs reported for the incorporation of unnatural amino acids^{1, 6, 7} (after appropriate anticodon conversion using the steps reported here) with ribo-Q1 it will be possible to encode more than 200 amino acid combinations in recombinant proteins.

### Experimental (Methods Summary)

Methods for cloning, site-directed mutagenesis and library construction are described in the Supplementary Materials. Ribosome libraries were screened for quadruplet suppressors using a modification of the strategy to discover ribo-X⁹.

E. coli genehogs or DH10B were used in all protein expression experiments using LB medium supplemented with appropriate antibiotics and unnatural amino acids. Proteins were purified by affinity chromatography using published standard protocols. Translational fidelity of evolved O-ribosomes was measured by mis-incorporation of ³⁵S-labelled cysteine ⁹. Briefly, GST-MBP was produced by the O-ribosome in the presence of ³⁵S-cysteine. The protein was purified, cleaved with thrombin, which cleaves the linker between GST and MBP, and analysed by SDS-PAGE and phospho-imaging. A modified Dual-luciferase assay was used to measure the fidelity of translation of O-ribosomes⁹. Luminescence from a luciferase mutant containing an inactivating missense mutation in this assay is a measure of translational inaccuracy of the ribsome. The DLR was translated by the O-ribosome, extracted in the cold and luciferase activity measured using the Dual-Luciferase Reporter Assay System (Promega).

LC/MS/MS of proteins was performed by NextGen Science (Ann Arbor, USA). Proteins were excised from Coomassie stained SDS-PAGE gels, digested with trypsin and analysed by LC/MS/MS. Total protein mass was obtained by ESI-MS; purified protein was dialysed into 10 mM ammonium bicarbonate pH 7.5, mixed 1:1 with 1% formic acid in 50% methanol and total mass determined in positive ion mode.

Cyclization reactions were performed for 5 minutes at room-temperature on purified protein in 50 mM sodium phosphate pH 8.3 in the presence of 1 mM ascorbic acid, 1 mM CuSO4 and 2 mM bathophenathroline. Details of all methods can be found in the Supplementary Materials.

### Definitions

The term ‘comprises’ (comprise, comprising) should be understood to have its normal meaning in the art, i.e. that the stated feature or group of features is included, but that the term does not exclude any other stated feature or group of features from also being present.

### BRIEF DESCRIPTION OF THE FIGURES

FIG. 1. Selection and characterization of orthogonal quadruplet decoding ribosomes. a. Mutations in quadruplet decoding ribosomes form a structural cluster close to the space potentially occupied by an extended anticodon tRNA. Selected nucleotides are shown in red. b. Ribo-Qs substantially enhances the tRNA decoding of quadruplet codons. The tRNA^{ser2}_{UCCU}-dependent enhancement in decoding AGGA codons in the O-cat (AGGA103, AGGA146) gene was measured by survival on increasing concentrations of chloramphenicol (Cm). c. As in b, but measuring CAT enzymatic activity directly by thin-layer chromatography acetylated chloramphenicol (AcCm). ribo-X (Rx), ribo-Q1-4 (Q1-Q4) and the O-ribosome (O)

FIG. 2. Enhanced incorporation of unnatural amino acids in response to amber and quadruplet codons with ribo-Q1. a. Ribo-Q1 incorporates Bpa (p-benzoyl-L-phenylalanine) as efficiently as ribo-X. The entire gel is shown in FIG. 10. b. Ribo-Q1 enhances the efficiency AzPhe (p-azido-L-phenylalanine) in response to the AGGA quadruplet codon using AzPheRS*/tRNA_{UCCU}. The gel showing the ratio of GST-MBP to GST as well as MS/MS spectra of the single and double AzPhe incorporations are shown in FIG. 11. (UAG)ₙ or (AGGA)ₙ describes the number of amber or AGGA codons (n) between gst and malE.

FIG. 3. Encoding an azide and an alkyne in a single protein via orthogonal translation. a. Expression of GST-CaM-His₆ (a glutathione-S-transferase calmodulin his6 fusion) containing two unnatural amino acids. An orthogonal gene producing a GST-CaM-His₆ fusion that contains an AGGA codon at position 1 and an amber codon at position 40 of calmodulin (CaM)) was translated by ribo-Q1 in the presence of AzPheRS*/tRNA_{UCCU} and MbPyIRS/tRNA_{CUA}. The entire gel is shown in FIG. 17. b. LC/MS/MS analysis of the incorporation of two distinct unnatural amino adds into the linker region of GST-MBP. (2 is denoted as Y* and 4 as K*).

FIG. 4. Genetically directed cyclization of calmodulin via a Cu(I)-catalyzed Huisgens [3+2]-cycloaddition. a. Structure of calmodulin indicating the sites of incorporation of 2 and 4 and their triazole product. Image created using Pymol (www.pymol.org) and pdb-file 4CLN. b. GST-CaM-His₆ 1AzPhe 149CAK specifically cyclizes with Cu(I)-catalyst. AzPhe is 2, Tyr is tyrosine, BocK is 3 and CAK is 4. Lanes 1 and 2 are from a separate gel c. LC/MS/MS confirms the triazole formation. The MS/MS spectra of a doubly charged peptide containing the crosslink (m/z=1226.6092, which is within 1.8 ppm of the mass expected for cross-linked peptide).

FIG. 5. Strategy for the synthesis of an orthogonal genetic code. Combining the two mutually orthogonal pairs (MbPyIRS/MbtRNA_{CUA} and MjAzPheRS*/tRNA_{UCCU}) with evolved orthogonal ribosomes (Ribo-Q) creates a system that is able to decode the UAG and AGGA codons on an orthogonal mRNA (O-mRNA) to produce a protein that contains two distinct unnatural amino acids at genetically encoded sites. UAG is decoded as 4 (CAK) or 3 (BocLys) by MbPyIRS/MbtRNA_{CUA} while AGGA is decoded as 2.

FIG. 6. Evolving an orthogonal quadruplet decoding ribosome. The natural ribosome (gray) and the progenitor orthogonal ribosome (green) utilize tRNAs with triplet anticodon to decode triplet codons in both wt—(black) and orthogonal—(purple) mRNAs, respectively. The decoding of quadruplet codons with extended anticodon tRNAs (red) is of low efficiency (light gray arrows) on both ribosomes. Synthetic evolution of the orthogonal ribosome leads to an evolved scenario in which a mutant (orange patch) orthogonal ribosome more efficiently decodes quadruplet codons on orthogonal mRNAs using extended anticodon tRNAs. Decoding of extended anticodon tRNAs on natural mRNAs is unaffected because the orthogonal ribosome does not read natural mRNAs and the natural ribosome is unaltered.

FIG. 7. Comprehensive mutagenesis of the ribosome decoding centre. A. Structure of the ribosomal small subunit with bound tRNAs and mRNAs. tRNA anticodon stem loops are bound to A site (yellow), P site (cyan), and E site (dark blue). The mRNA is shown in purple. 16S ribosomal RNA is shown in green and ribosomal proteins in gray. The 118 residues in the decoding centre, targeted for mutation in the 11 libraries, are shown in orange (This figure was created using Pymol v0.99 (www.pymol.org) and PDB ID 2J00). B. Secondary structure of the E. coli 16S ribosomal RNA (www.rna.ccbb.utexas.edu). The nucleotides targeted for mutation are shown colored orange.

FIG. 8. Ribo-Q enhances the tRNA dependent decoding of different quadruplet codons. Ribo-X, Ribo-Q1-4 and the O-ribosome were produced from pRSF-O-rDNA vectors. The tRNAser2UCUA-dependent enhancement in decoding UAGA codons in the O-cat (UAGA103, UAGA146), the tRNAser2AGGG-dependent enhancement in decoding CCCU codons in the O-cat (CCCU103, CCCU146), and the tRNAser2UCUU-dependent enhancement in decoding AAGA codons in O-cat (AAGA146) was measured by survival on increasing concentrations of chloramphenicol. pRSF-O-rDNA vectors and corresponding O-cat vectors were co-transformed into GeneHogs cells. Transformed cells were recovered for 1 h in SOB medium containing 2% glucose and used to inoculate 200 ml of LB-GKT (LB medium with 2% glucose, 25 μg ml⁻¹ kanamycin and 12.5 μg ml⁻¹ tetracycline). After overnight growth (37° C., 250 r.p.m., 16 h), 2 ml of the cells were pelleted by centrifugation (3,000 g), and washed three times with an equal volume of LB-KT (LB medium with 12.5 μg ml⁻¹ kanamycin and 6.25 μg ml⁻¹ tetracycline). The resuspended pellet was used to inoculate 18 ml of LB-KT, and the resulting culture incubated (37° C., 250 r.p.m. shaking, 90 min). To induce expression of plasmid encoded O-rRNA, 2 ml of the culture was added to 18 ml LB-IKT (LB medium with 1.1 mM isopropyl-D-thiogalactopyranoside (IPTG), 12.5 μg ml⁻¹ kanamycin and 6.25 μg ml⁻¹ tetracycline) and incubated for 4 h (37° C., 250 r.p.m.). Aliquots (250 μl optical density at 600 nm (OD600)=1.5) were plated on LB-IKT agar (LB agar with 1 mM IPTG, 12.5 μg ml⁻¹ kanamycin and 6.25 μg ml⁻¹ tetracycline) supplemented with 50 μg ml⁻¹ chloramphenicol and incubated (37° C., 40 h).

FIG. 9: The translation fidelity of evolved ribosomes is comparable to that of the natural ribosome. A. The translational error frequency for triplet decoding as measured by ³⁵S-cysteine misincorporation is indistinguishable for ribo-Q1, ribo-Q3-Q4, ribo-X, the unevolved orthogonal ribosome and the wild-type ribosome. GST-MBP was synthesized by each ribosome in the presence of ³⁵S-cysteine, purified on glutathione sepharose and digested with thrombin. The left panel shows a Coomassie stain of the thrombin digest. The un-annotated bands result primarily from the thrombin preparation. The right panel shows ³⁵S labeling of proteins in the same gel, imaged using a Storm Phosphorimager. Lanes 1-6 show thrombin cleavage reactions of purified protein derived from cells containing the indicated ribosome (with the ribosomal RNA produced from pSC101* constructs that drive rRNA from a P1P2 promoter) and either pO-gst-malE (for orthogonal ribosomes) or pgst-malE (for wild-type ribosomes). The size markers are pre-stained standards (Bio-Rad 161-0305). The error frequency per codon translated by the ribo-Q ribosomes as measured by this method was less than 1×10⁻³. Control experiments with the progenitor orthogonal ribosome, ribo-X and the wild-type ribosome allowed us to put the same limit on their fidelity. This limit compares favourably with previous measurements of error frequency using ³⁵S mis-incorporation (4×10⁻³ errors per codon) ³³B. The translational fidelity of ribo-Q1 in triplet decoding is comparable to that of the un-evolved ribosome, as measured by a dual-luciferase assay. In this system a C-terminal firefly luciferase is mutated at codon K529(AAA), which codes for an essential lysine residue. The extent to which the mutant codon is misread by tRNA^{Lys}(UUU) is determined by comparing the firefly luciferase activity resulting from the expression of the mutant gene to the wild-type firefly luciferase, and normalizing any variability in expression using the activity of the co-translated N-terminal Renilla luciferase. Previous work has demonstrated that measured firefly luciferase activities in this system result primarily from the synthesis of a small amount of protein that mis-incorporates lysine in response to the mutant codon²³, rather than a low activity resulting from the more abundant protein containing encoded mutations. In experiments examining the fidelity of ribo-Q1, lysate from cells containing pSC101*-ribo-Q1 and pO-DLR and its codon 529 variants were assayed. Control experiments used lysates from cells containing pSC101*-O-ribosome and pO-DLR and its codon 529 variants. C. The quadruplet decoding fidelity of ribo-Q is comparable to that of un-evolved ribosomes. Efficiencies were determined using a dual luciferase construct with an N-terminal Renilla and C-terminal Firefly luciferase (Ren-FF). The reporter was mutated to include a quadruplet AGGA codon in the linker between the two luciferases (Ren-AGGA-FF). Ren-AGGA-FF was transformed into DH10B cells along with a non-cognate anticodon Ser2A tRNA (UCUA or AGGG) and either ribo-Q or the O-ribosome. Readthrough efficiency for Ren-AGGA-FF was measured by taking the ratio of Firely luminescence/Renilla luminescence. This data was divided by the same Firefly/Renilla ratio when using the Ren-FF construct in the presence of tRNA (to normalize for effects of the tRNA on sites outside the AGGA codon under investigation). In order to obtain the level of decoding by these non-cognate tRNAs as a fraction of decoding by cognate tRNA, these data were compared with that obtained from the same experiment using a cognate Ser2A tRNA with the UCCU anti-codon. The data represent the average of at least 4 trials. The error bars represent the standard deviation. D Fourth base specificity in quadruplet decoding. E. coli DH10 B expressing the indicated combination of an O-ribosome, a chloramphenicol acetyltransferase gene under the control of an orthogonal rbs with a quadruplet codon at a permissive site and E. coli Ser2A tRNA_{UCCU} were scored for their ability to grow in the presence of increasing amounts of chloramphenicol. The fractional activity is the maximal Cm resistance of the cells relative to the combination containing a cognate codon in the mRNA and a particular o-ribosome.

FIG. 10: Ribo-Q1 enhances the efficiency of BpaRS/tRNA_{CUA}-dependent unnatural amino acid incorporation in response to single and double UAG codons, maintaining the enhanced amber decoding of ribo-X. In each lane an equal volume of protein purified from glutathione sepharose under identical conditions is loaded. Orthogonal ribosomes are produced from pSC101*-ribo-X, pSC101*-ribo-Q1. Bpa, p-benzoyl-L-phenylalanine (1). The BpaRS/tRNA_{CUA} pair is produced from pSUPBpa that contains six copies of MjtRNA_{CUA}. (UAG)ₙ describes the number of amber stop codons (n) between gst and malE in O-gst(UAG)ₙmalE or gst(UAG)ₙmalE. The ratio of GST-MBP to GST reflects the efficiency of amber suppression versus RF1 mediated termination. A part of this gel showing the band for full-length GST-MBP is shown in FIG. 2 of the main text.

FIG. 11: Ribo-Q1 enhances the efficiency of AzPheRS*/tRNA_{UCCU} unnatural amino acid incorporation in response to AGGA quadruplet codons. A. Ribo-Q1 is produced from pSC101*-ribo-Q1. AzPhe, 2.5 mM 2. The AzPheRS*/tRNA_{UCCU} pair is produced from pDULE AzPheRS*/tRNA_{UCCU} that contains a single copy of MjtRNA_{UCCU}. (AGGA)ₙ describes the number of quadruplet codons (n) between gst and malE in O-gst(AGGA)ₙmalE or gst(AGGA)ₙmalE. The ratio of GST-MBP to GST reflects the efficiency of frameshift suppression. A part of this gel showing the bands for full-length GST-MBP is shown in FIG. 2 of the main text. B & C. MS/ MS spectra of tryptic fragments incorporating one or two AzPhes respectively.

FIG. 12. MbPyIRS/MbtRNA_{CUA} and MjTyrRS/tRNA_{CUA} pairs are mutually orthogonal in their aminoacylation specificity. A. The decoding network of MbPyIRS/MbtRNA_{CUA} (lime) and MjTyrRS/tRNA_{CUA} (grey) and its unnatural amino acid incorporating derivatives. A unique unnatural amino acid is specifically recognized by each of the synthetases and used to aminoacylate its cognate tRNA. We asked whether the MbPyIRS/tRNA_{CUA} pair^{4, 5, 34} and MjTyrRS/tRNA_{CUA} pair are mutually orthogonal in their aminoacylation specificity.

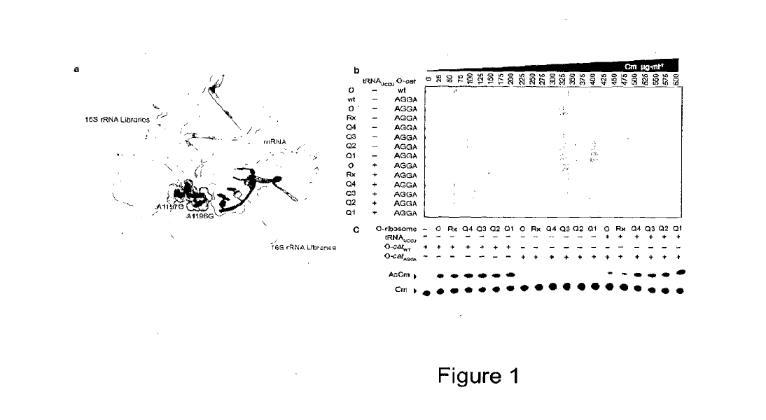

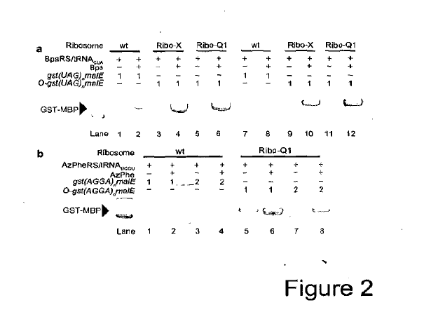

Our experiments demonstrate that there is no cross-acylation (grey arrows) between the two aminoacyl-tRNA synthetase/tRNA_{CUA} pairs (as shown by decoding the amber codon in myo4TAGHis₆ using the different combinations of synthetases and tRNAs, see below). However, both tRNAs direct the incorporation of their amino acid in response to the amber codon. B. E. coli DH10B were transformed with pMyo4TAG-His_{6,} a plasmid holding the gene for sperm whale myoglobin with an amber codon at position 4 and a C-terminal hexahistidine tag and an expression cassette for either MbtRNA_{CUA} or MjtRNA_{CUA}. MbPyIRS or MjTyrRS were provided on pBKPyIS or pBKMjTyrRS, respectively. Cells expressing MbPyIRS received 10 mM 3 (BocLys) as a substrate for the synthetase. Myoglobin-His₆ produced by the cells was purified by Ni²⁺-affinity chromatography, analysed by SDS-PAGE and detected with Coomassie stain or Western blot against the His₆-tag.

FIG. 13. Genetically encoding 2 in response to a quadruplet codon. A. MjAzPheRS aminoacylates its cognate amber suppressor tRNA_{CUA} with 2. To differentiate the codons that the two mutually orthogonal tRNAs decode and to create a pair for the incorporation of an unnatural amino acid in response to a quadruplet codon, we altered the anticodon of MjtRNA_{CUA} from CUA to UCCU to create MjtRNA_{UCCU}. After this, the resulting tRNA_{UCCU} is no longer a substrate of the parent MjAzPheRS. To create a version of AzPheRS-7 that aminoacylates MjtRNA_{UCCU} we identified six residues (Y230, C231, P232, F261, H283, D286) in the parent synthetase that recognize the anticodon of the tRNA³⁵ and mutated these residues to all possible combinations, creating a library of 10⁸ possible synthetase mutants. To select for AzPheRS mutants that specifically aminoacylate MjtRNA_{UCCU} we created a chloramphenicol acetyl transferase reporter (pREP JY(UCCU), derived from pREP YC-JYCUA³²), which contains the four base codon AGGA at position 111, a site permissive to the incorporation of a range of amino acids. In the absence or presence of AzPheRS/MjtRNA_{UCCU} this reporter confers resistance to chloramphenicol at low levels (30-50 μg ml⁻¹). We selected synthetase variants on 150 μg ml⁻¹ of chloramphenicol that, in combination with MjtRNA_{UCCU}, specifically direct the incorporation of 2 in response to the AGGA codon on pREP JY(UCCU). We characterized 24 synthetase/tRNA_{UCCU} pairs by their chloramphenicol resistance in the presence of 2 and pREP JY(UCCU). The seven best synthetase/tRNA_{UCCU} combinations confer a chloramphenicol resistance of 250-350 μg ml⁻¹ on cells containing 2 and pREP JY(UCCU) (FIG. 14). In the absence of the 2, we observe only background levels of resistance (30 μg ml⁻¹) for several synthetases indicating that the synthetase/MjtRNA_{UCCU} pairs specifically direct the incorporation of 2 in response to the quadruplet codon AGGA. Sequencing these seven clones revealed similar but non-identical mutations (FIG. 14). B. Library design. Structure of MjTyrRS (grey) bound to its cognate tRNA (orange). Residues of the synthetase that recognize the anticodon and which are mutated in the library, as well as bases of the natural anticodon (G34, U35, A36) are shown in blue (Figure created using Pymol, www.pymol.org, and pdb-file 1J1U). C. The production of full-length myoglobin from myo4(AGGA)-his₆ by the AzPheRS*-2/MjtRNA_{UCCU} pair is dependent on the presence of 2. In the remainder of the text we refer to MjAzPheRS*-2 as MjAzPheRS* for simplicity. MjAzPheRS*/tRNA_{UCCU} efficiently suppress an AGGA codon placed into the myoglobin gene. E. coli DH10B were transformed with pMyo4TAG-His₆ or pMyo4AGGA-His₆, a plasmid holding the gene for sperm whale myoglobin with an amber or an AGGA codon at position 4, respectively, and a C-terminal hexahistidine tag and an expression cassette for either MjtRNA_{CUA} or MjtRNA_{UCCU}. MjAzPheRS or MjAzPheRS* were provided on pBKMjAzPheRS or pBKMjAzPheRS*, respectively. Cells received 2.5 mM 2 as a substrate for the synthetase. Myoglobin-His₆ produced by the cells was purified by Ni²⁺-affinity chromatography, analysed by SDS-PAGE and detected with Coomassie stain. D. MjAzPheRS*/tRNA_{UCCU} decodes AGGA codons specifically with 2. The incorporation of 2 into myoglobin-His₆ purified from cells expressing Myo4(AGGA) and MjAzPheRS*/tRNA_{UCCU} in the presence of 2.5 mM 2 was analysed by ESI-MS. The mass of the observed peak (18457.75 Da) corresponds to the calculated mass of myoglobin containing a single 2 (18456.2 Da).

FIG. 14: Amino acid dependent growth of selected MjAzPheRS* variants. E. coli DH10B were co-transformed with isolates from a library built on pBK MjAzPheRS-7 and pREP JY(UCCU) (coding for MjtRNA_{UCCU} and chloramphenicol acetyltransferase with an AGGA codon at position D111). Cells were grown in the presence or absence of 1 mM 2 for 5 h and pronged onto LB agar plates containing 25 μg ml⁻¹ kanamycin, 12.5 μg ml⁻¹ tetracycline and the indicated concentration of chloramphenicol with or without the unnatural amino acid. Plates were photographed after 18 h at 37° C. Sequencing of mutations for incorporating tyrosine, 2 and propargyl-L-tyrosine (FIG. 15) in response to the AGGA codon reveals clones with common mutations Y230K, C231 K and P232K, but divergent mutations at positions F261, H283 and D286. This suggests that amino acids 230, 231 and 232 confer affinity and specificity for the anticodon, and that 261, 283 and 286 may couple the identity of the anticodon to the amino acid identity.

FIG. 15: Amino acid dependent growth of selected MjPrTyrRS* variants. E. coli DH10B transformed as in FIG. 14 using isolates from a library built on MjPrTyrRS and tested for unnatural amino acid dependent growth. Mutations relative to MjPrTyrRS are given in the table below.

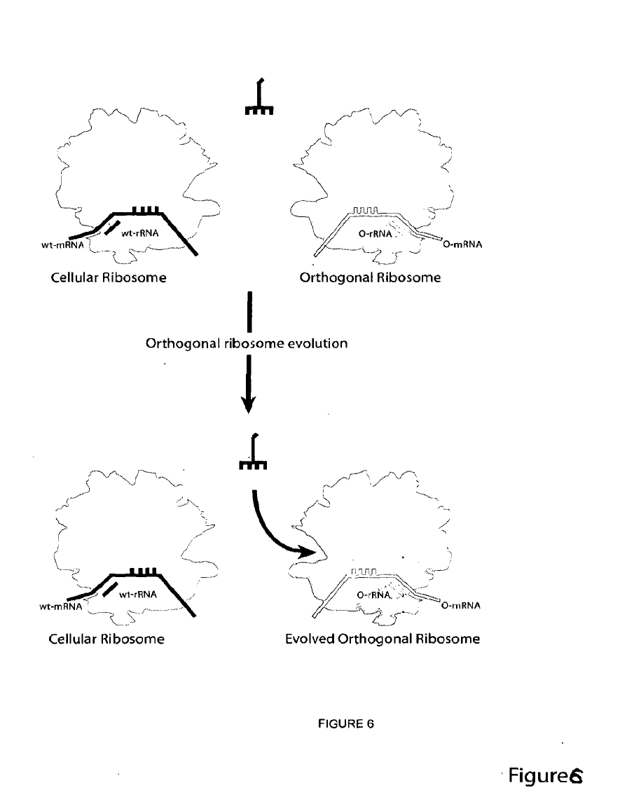

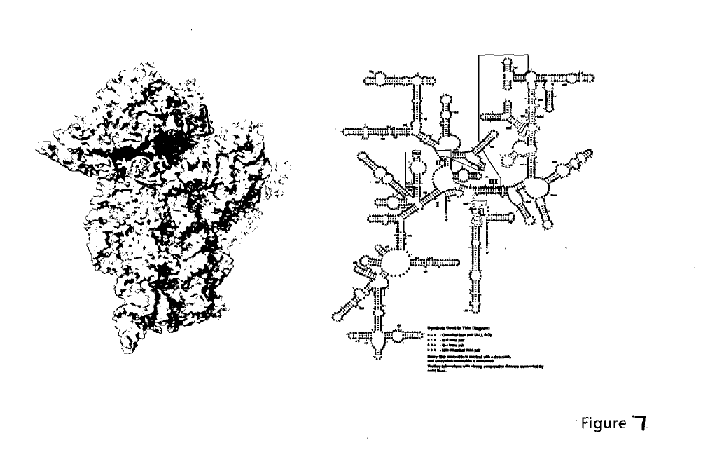

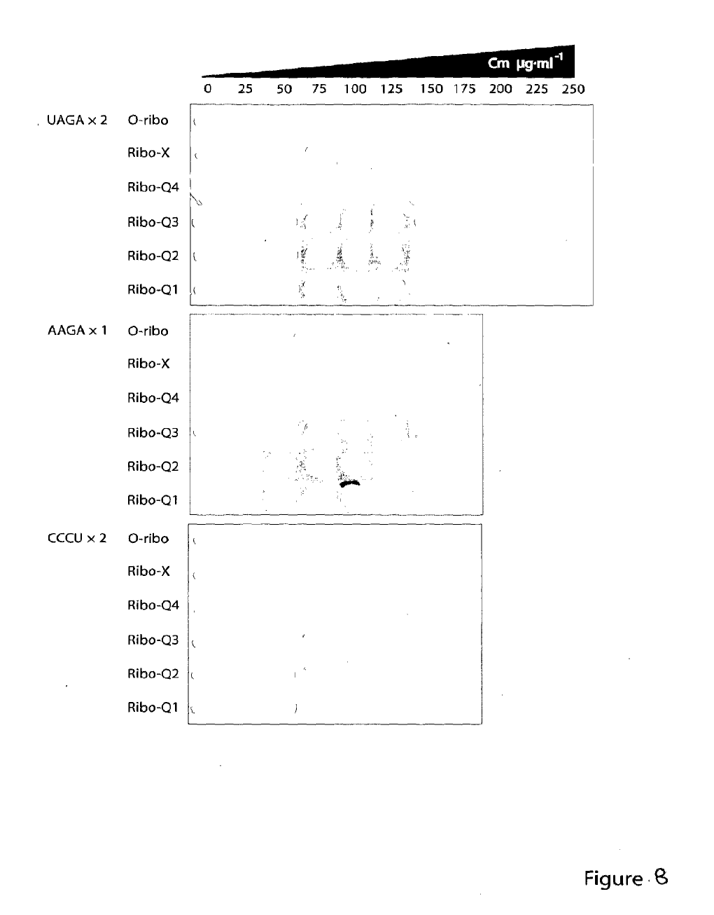

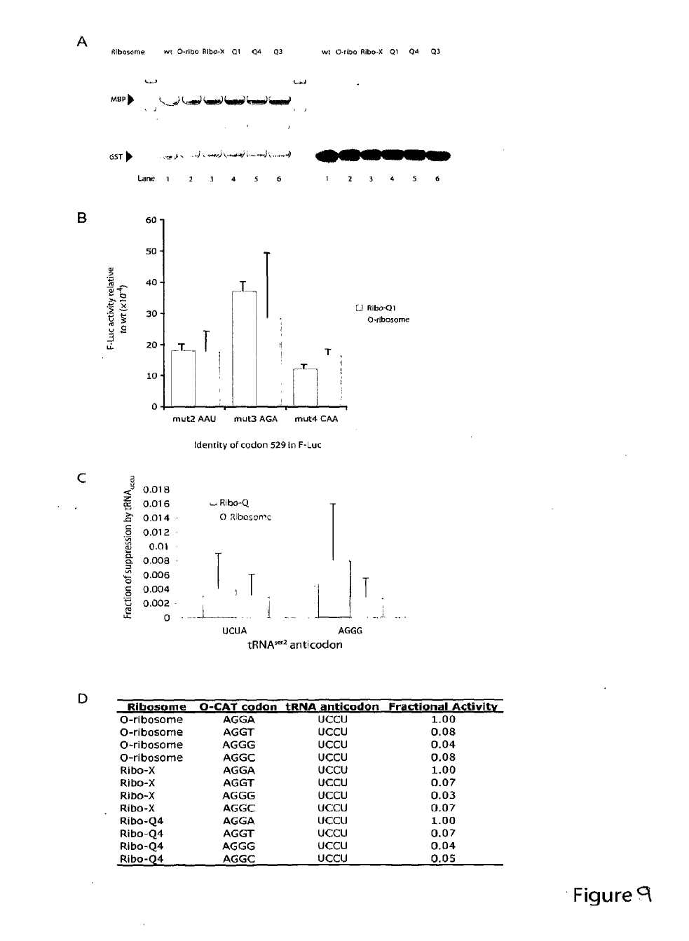

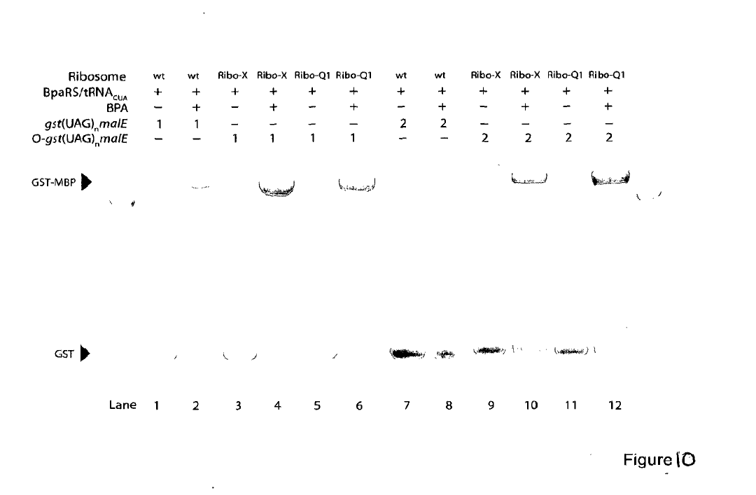

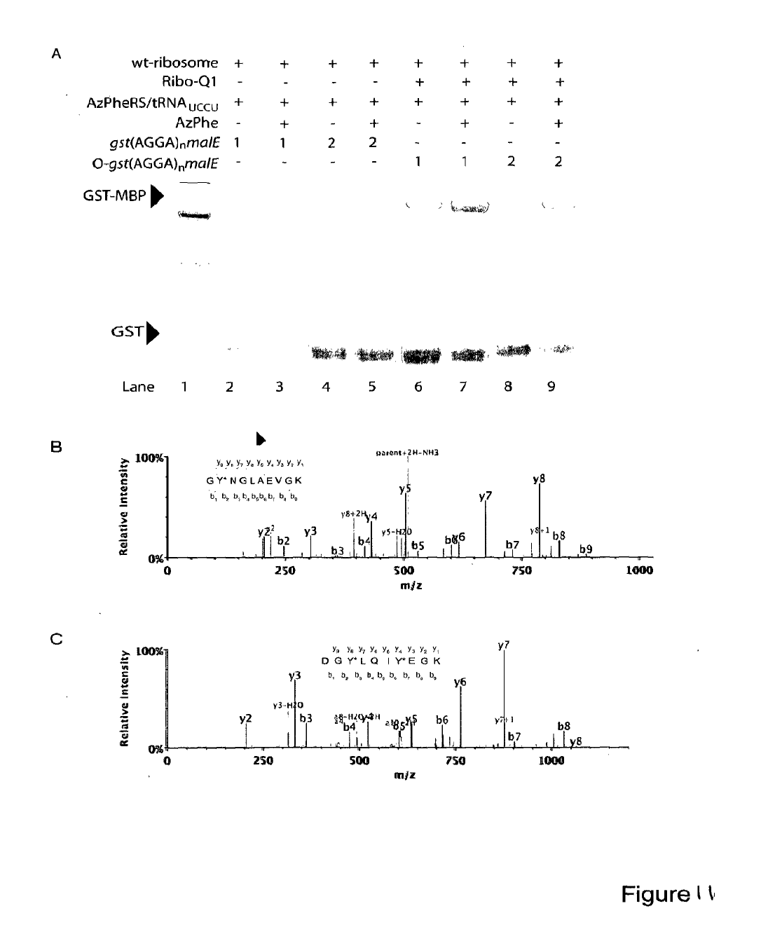

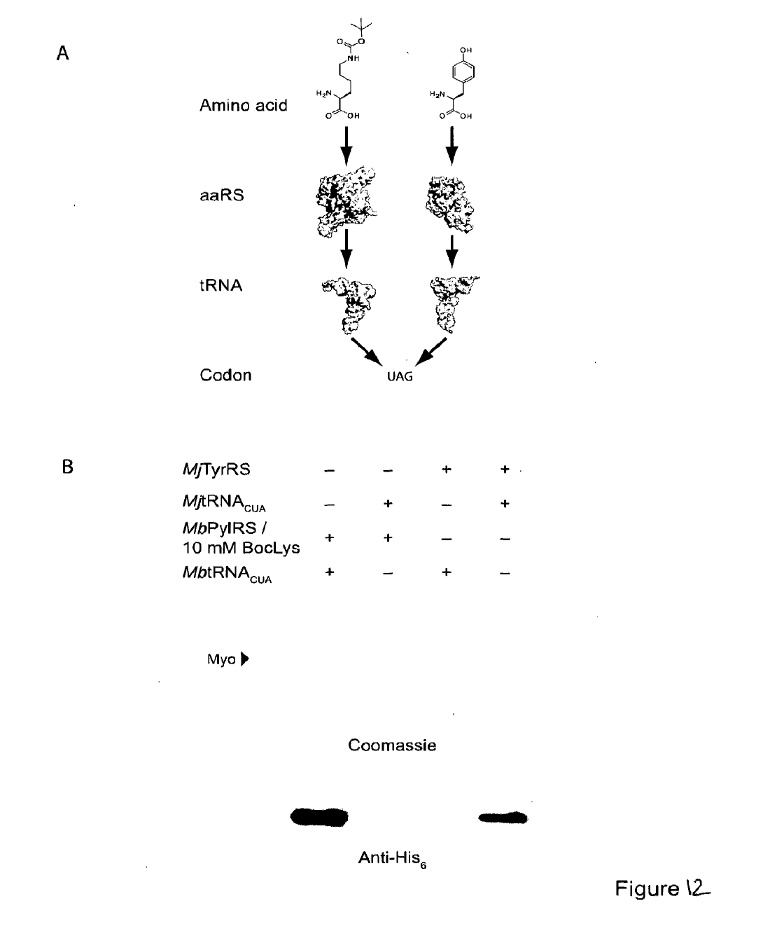

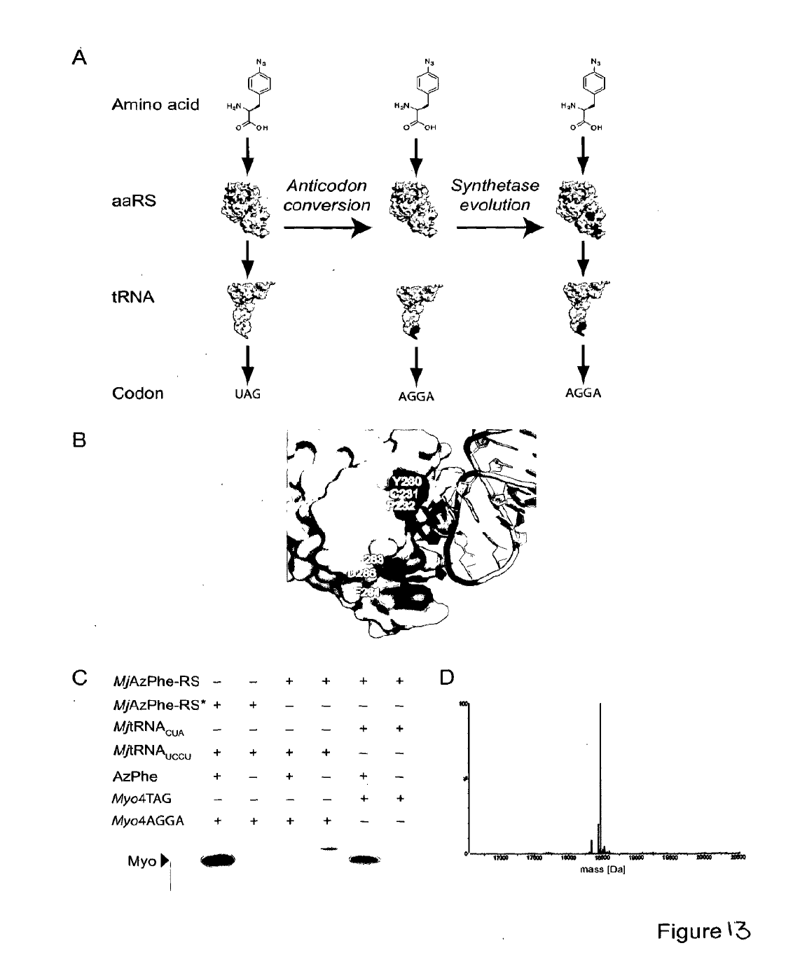

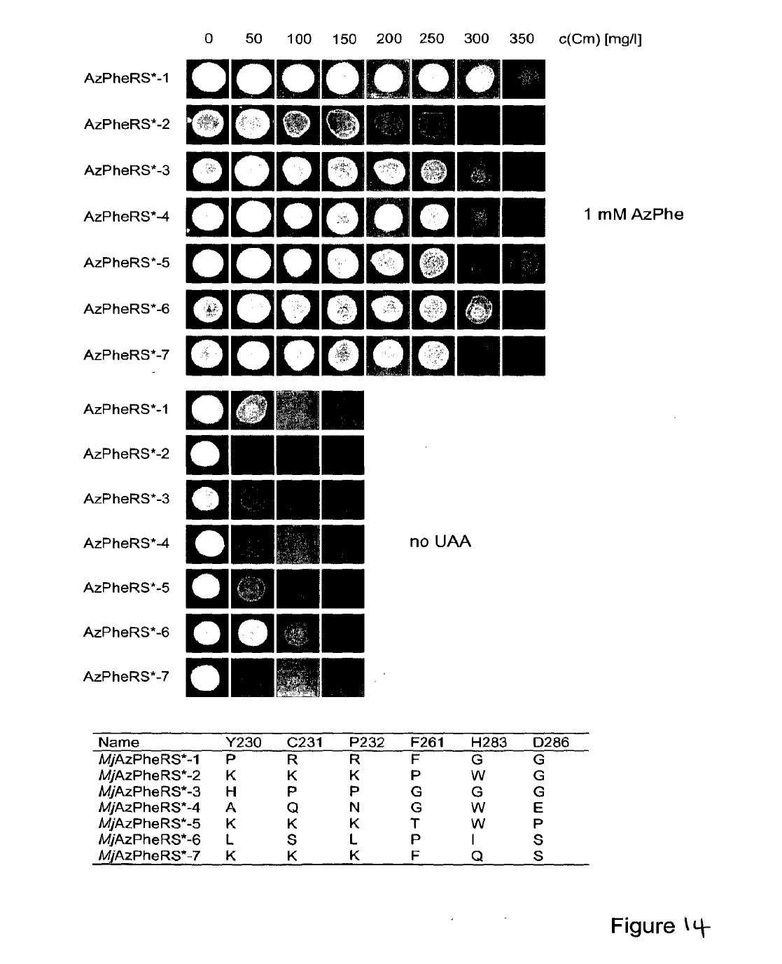

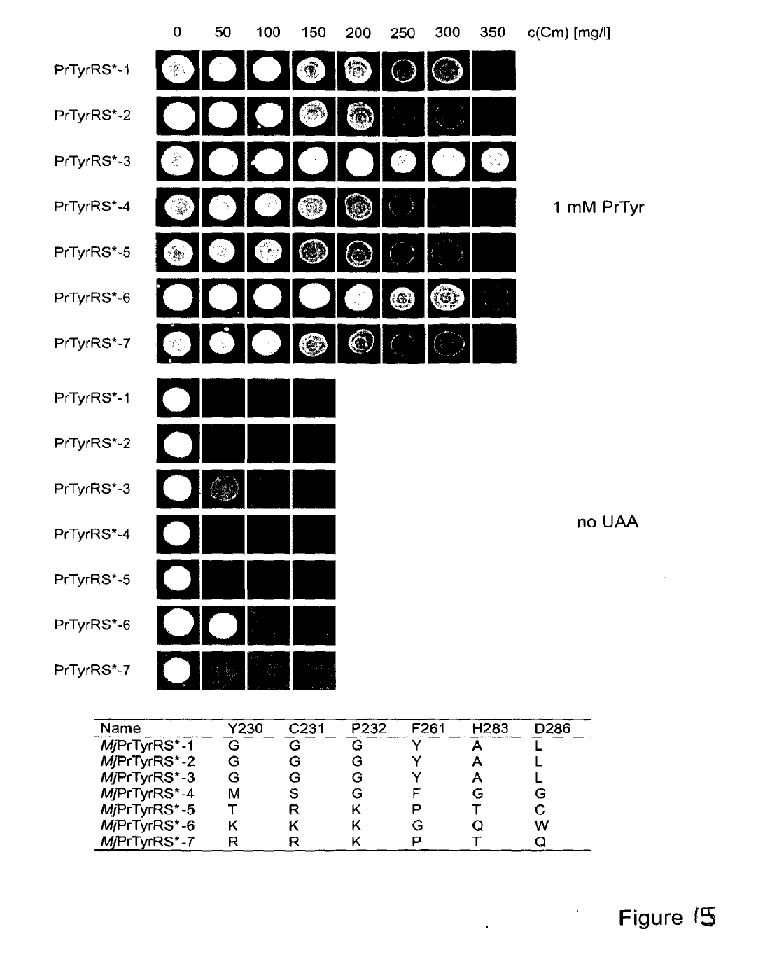

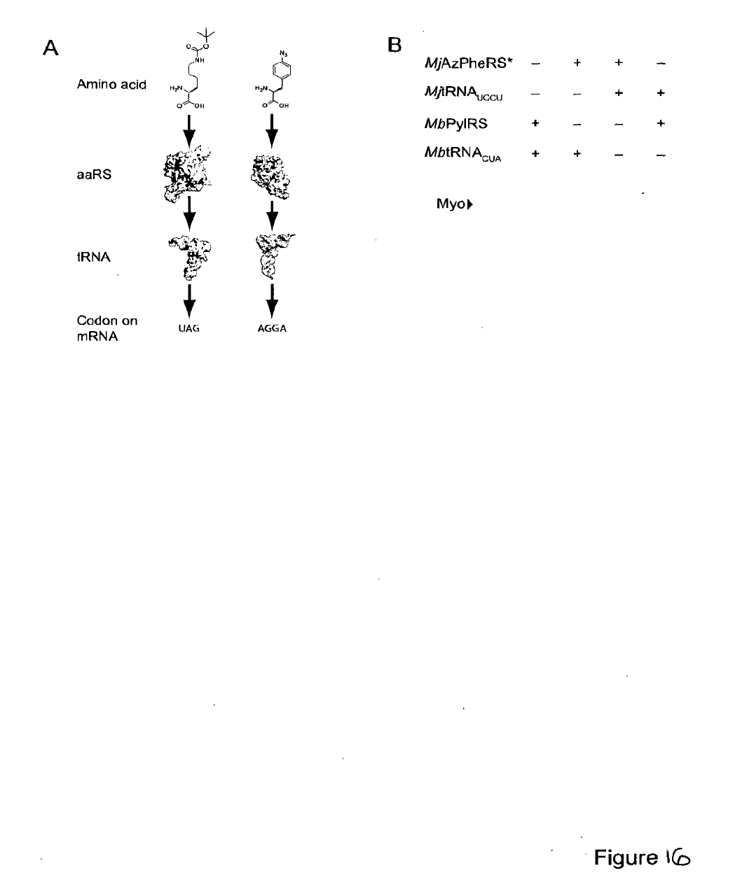

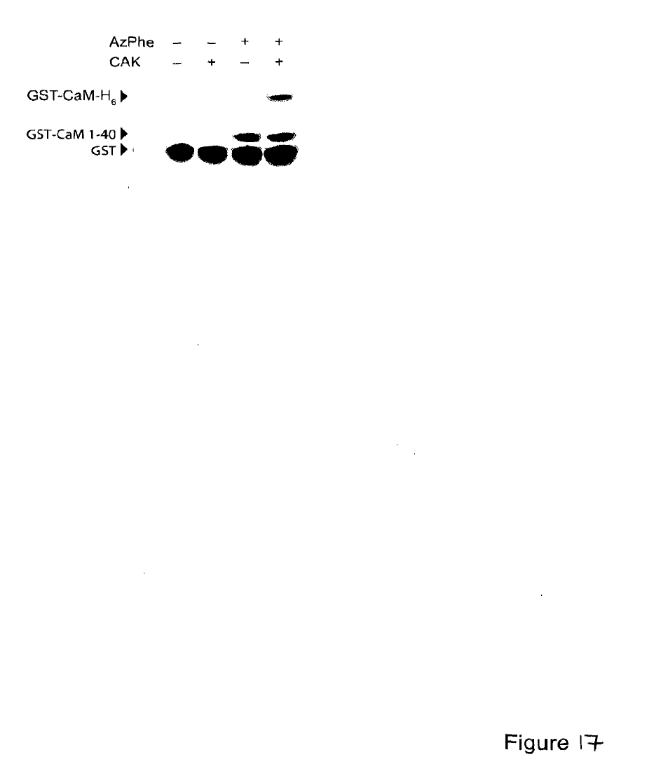

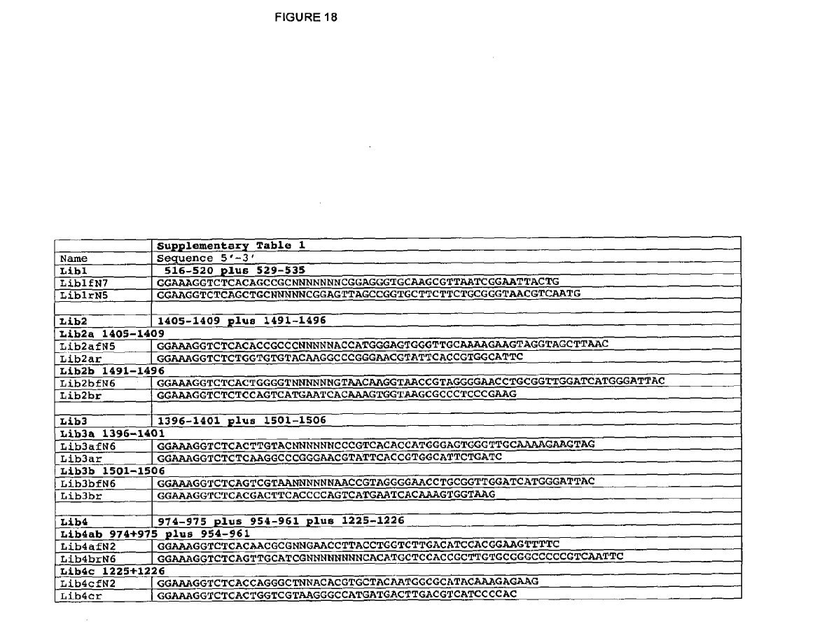

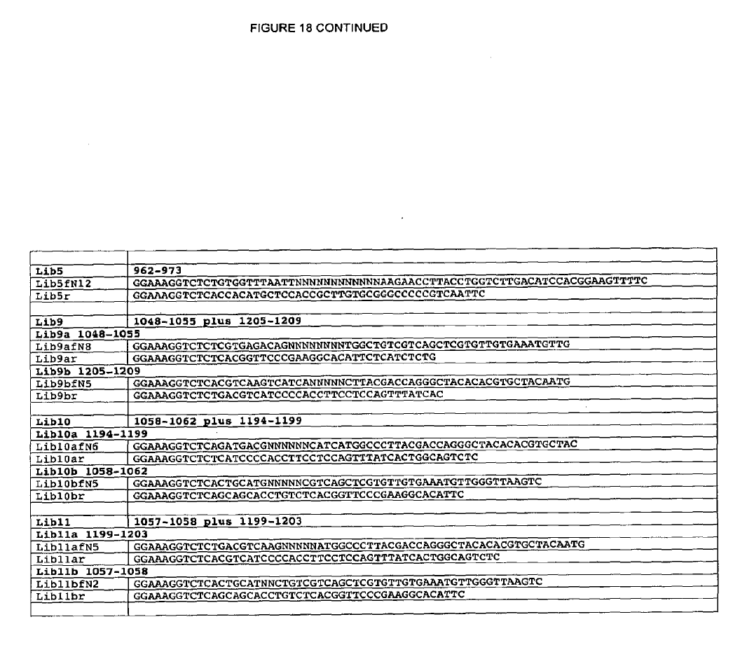

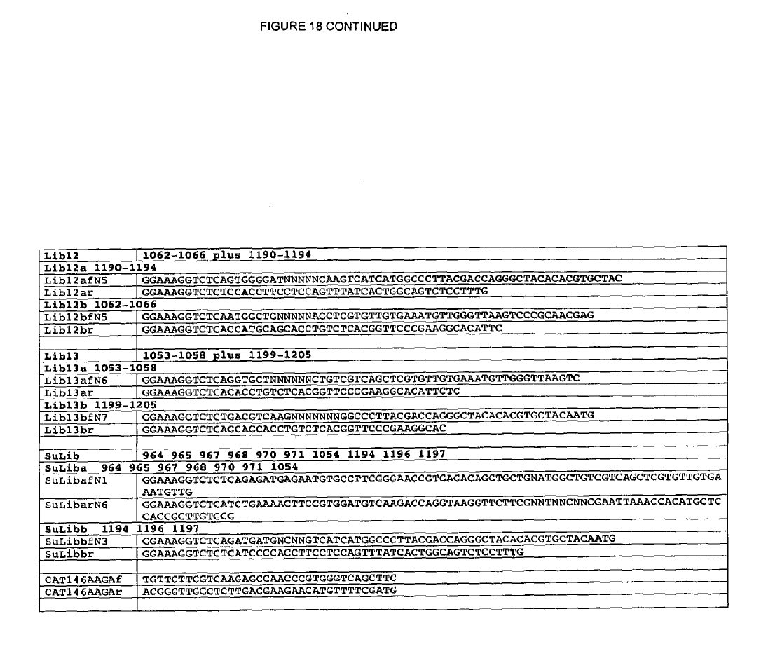

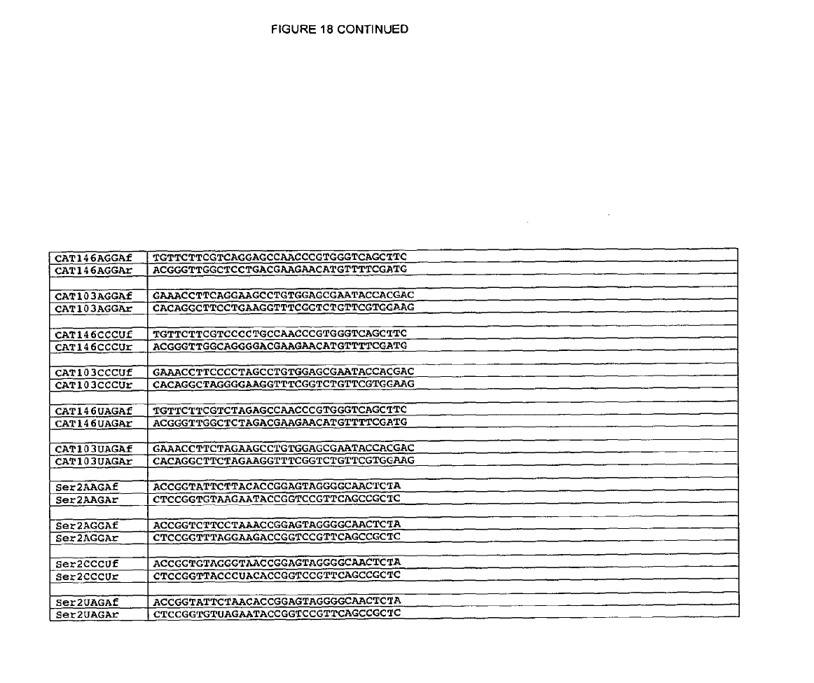

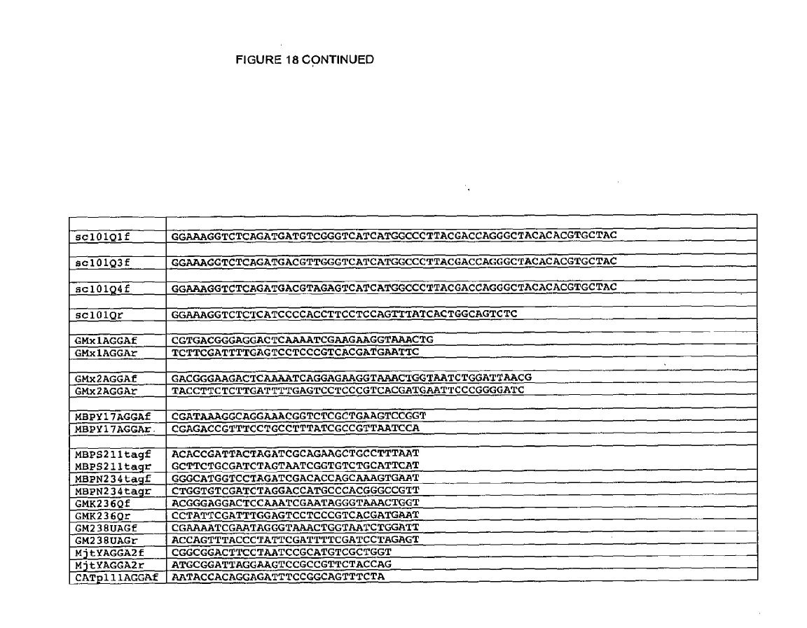

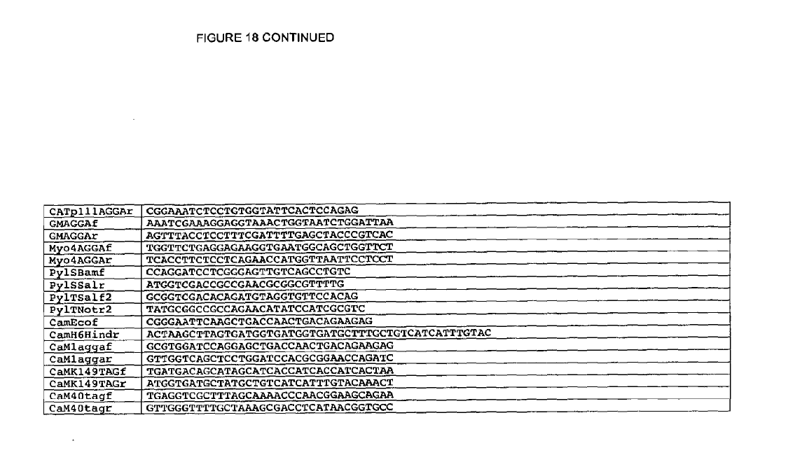

FIG. 16: The MbPyIRS/MbtRNA_{CuA} and MjAzPheRS*/tRNA_{UCCU} pairs incorporate distinct unnatural amino acids in response to distinct unique codons. A. The two orthogonal pairs (MbPyIRS/MbtRNA_{CUA} and MjAzPheRS*/tRNA_{UCCU} ) decode two distinct codons in the mRNA (UAG and AGGA) with two distinct amino acids (N6-[(tert.-butyloxy)carbonyl]-L-lysine and 2). MbPyIRS does not aminoacylate MjtRNA_{UCCU} and MbtRNA_{CUA} is not a substrate for MjAzPheRS*. B. Suppression of a cognate codon at position 4 in the gene of sperm whale myoglobin by different combinations of MbPyIRS/MbtRNA_{CUA} and MjAzPheRS*/tRNA_{UCCU}. E. coli DH10B were transformed with pMyo4TAG-His₆ or pMyo4AGGA-His₆ as described in FIG. 6C. Cells were provided with MbPyIRS (on pBKPyIS) or MjAzPheRS* (on pBKMjPheRS*) and 2.5 mM N6-[(tert.-butyloxy)carbonyl]-L-lysine or 5 mM 2, respectively. Myoglobin-His₆ produced by the cells was purified by Ni²⁺-affinity chromatography, analysed by SDS-PAGE and detected with Coomassie stain. We see weak incorporation in response to the UAG codon using the MbPyIRS pair. This incorporation is independent of the presence of MjAzPheRS* and results from a low level background acylation of the tRNA by E. coli synthetases in rich media, as previously observed.

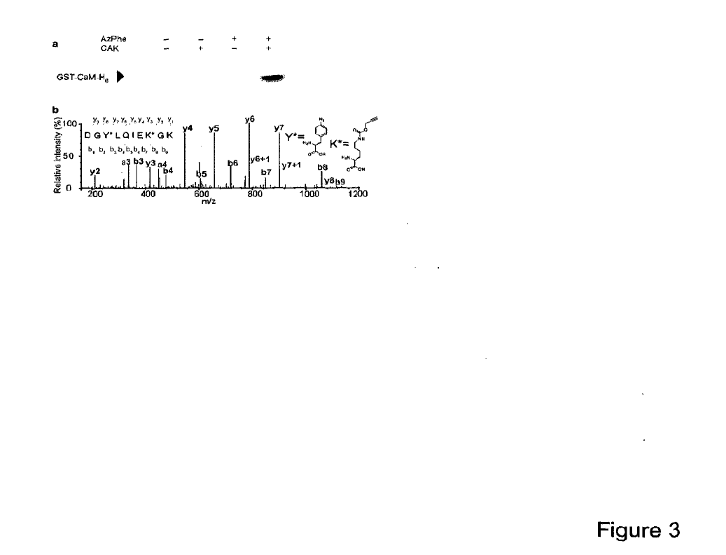

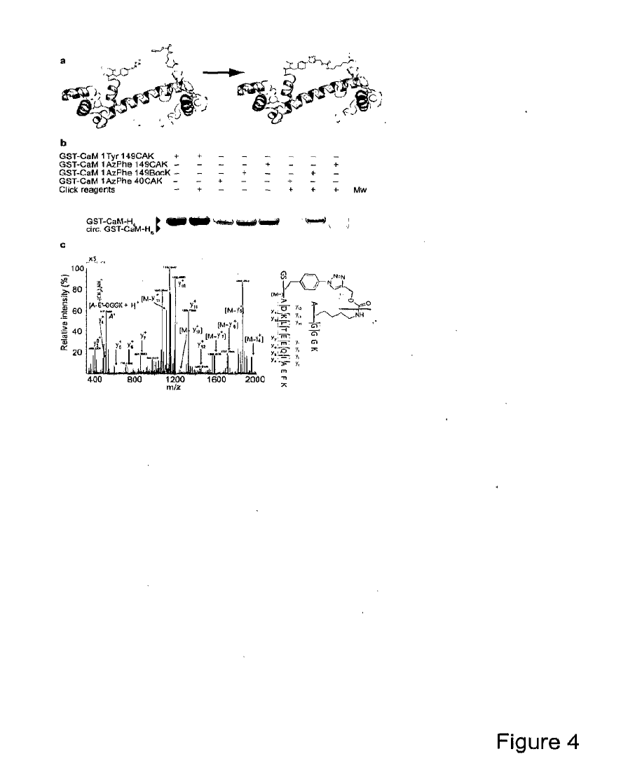

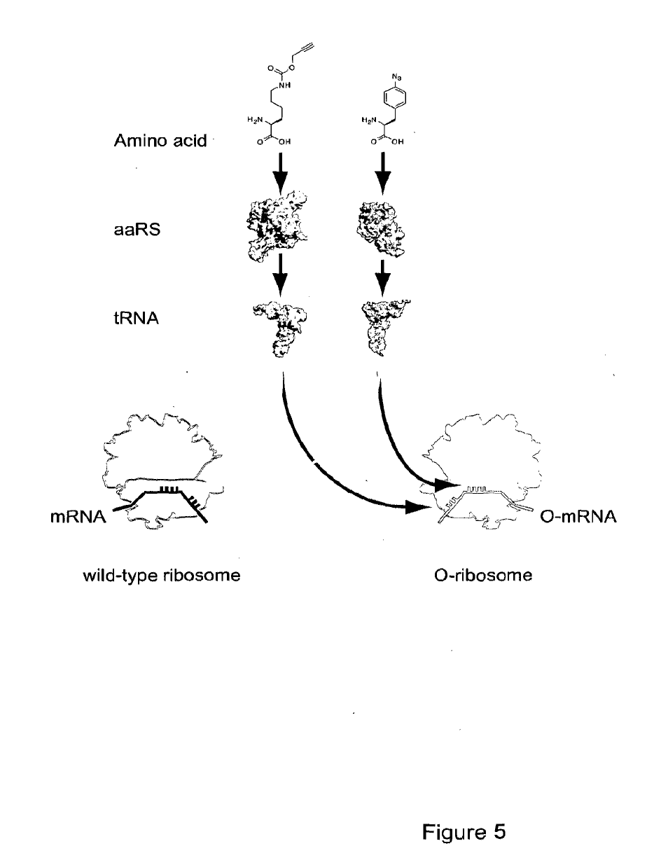

FIG. 17: Encoding an azide and an alkyne in a single protein via orthogonal translation. A. Expression of GST-COM-His₆ containing two unnatural amino acids. E. coli DH10B were transformed with four plasmids: pCDF PyIST (expressing MbPyIRS and MbtRNA_{CUA}), pDULE AzPheRS* tRNA_{UCCU} (encoding MjAzPheRS*/tRNA_{UCCU}), pSC101* ribo-Q1 and p-O-gst-CaM-His₆ 1AGGA 40UAG (a GST-CaM-His₆ fusion translated by the orthogonal ribosome that contains an AGGA codon at position 1 and an amber codon at position 40 of calmodulin (CaM)). Cells were grown in LB medium containing antibiotics to maintain the plasmids and 2.5 mM 4 and/or 5 mM 2 as indicated. Cells were harvested, lysed and the protein purified on GSH-beads. Bound protein was eluted with 10 mM GSH in PBS and analysed by SDS-PAGE. A part of this gel is shown in FIG. 3 of the main text. Full-length protein was produced by this method with yields of upto 0.5 mg/L

FIG. 18 shows Supplementary Table 1: Oligonucleotides used in this study.

The invention is now described by way of example. These examples are intended to be illustrative, and are not intended to limit the appended claims.

### EXAMPLES

### Plasmid Construction

Previously described gst-MalE protein expression vectors pgst-malE and pO-gst-malE⁹, are translated by wild type and orthogonal ribosomes respectively. These vectors were used as templates to construct variants containing one or two quadruplet codons in the linker region between the gst and malE open reading frame.

To create vectors containing a single AGGA quadruplet codon between gst and malE (pgst(AGGA)malE and pO-gst(AGGA)malE) the Tyr codon, TAC, in the linker between gst and malE was changed to AGGA by Quikchange mutagenesis (Stratagene), using the primers GMx1AGGAf and GMx1AGGAr (all primers used in this study are listed in Supplementary Table 1). For double AGGA mutants we additionally mutated the fourth codon in malE from GAA to AGGA by quick change PCR, with the primers GMx2AGGAf and GMx2AGGAr to create the vectors pgst(AGGA)₂malE and pO-gst(AGGA)₂malE. The vector pO-gst-malE(Y252AGGA) used for protein expression for mass spectrometry, in which the codon for Y17 of MBP was mutated to AGGA, was created by Quikchange mutagenesis (Stratagene) using the primers MBPY17AGGAf and MBPY17AGGAr.

To create vectors for constitutive production of the selected O-ribosomes the mutations in pRSF-OrDNA that confer the quadruplet decoding capacity on the orthogonal ribosome were transferred to pSC101 based O-rRNA expression vectors. pSC101*-ribo-X was used as a template and the mutations in 16S rDNA were introduced by enzymatic inverse PCR using the primers sc101Qr and sc101Q1f (for Ribo-Q1), sc101Q3f (forRibo-Q3) and sc101Q4f (for Ribo-Q4).

pDULE AzPheRS* tRNA_{UCCU} (containing the gene for MjtRNA_{UCCU} and MjAzPheRS*, each under the control of the Ipp promoter) was created by changing the anticodon of the MjtRNA_{CUA} to UCCU by Quikchange and replacing the ORF of the MjBPA-RS with MjAzPheRS*-2 via ligation of the MjAzPheRS*-2 gene, obtained by cutting pBK MjAzPheRS*-2 with the restriction enzymes Ndel and Stul, into the same sites on pDULE MjBPARS MjtRNA_{UCCU}. pCDF PyIST (a plasmid expressing MbPyIRS and MbtRNA_{CUA} from constitutive promoters) was created by cloning PCR products containing expression cassettes for MbPyIRS and MbtRNAcuA into the BamHI and SaLI or the SaLI and NotI sites of pCDF DUET-1 (Novagen). The PCR products were obtained by amplifying the relevant regions of pBK PyIRS and pREP PyIT.

Plasmid encoding a fusion of GST and CaM were created by replacing the ORF of MBP in p-O-gst-malE with human CaM. The gene for CaM was amplified by PCR from pET3-CaM (a kind gift from K. Nagai) using primers CamEcof and CamH6Hindr (adding a C-terminal His₆-tag) and cloned into the EcoRI and HindIII sites of pO-gst-malE. Methionine-1 of CaM was mutated to AGGA by a subsequent round of Quikchange mutagenesis using primers CaM1aggaf and CaM1aggar (simultaneously removing part of the linker between GST and CaM). In a second round of mutagenesis an amber codon was introduced at position 149 using primers CaMK149TAGf and CaMK149TAGr. To create a sterically hindered control the amber codon was inserted at position 40 instead using primers CaM40tagf and CaM40tagr.

### Construction of Ribosome Libraries and Quadruplet Decoding Reporters

11 different 16S rDNA libraries were constructed by enzymatic inverse PCR^{8, 31} using pTrcRSF-O-ribo-X as a template. The resulting pRSF-O-rDNA libraries mutate between 7 and 13 nucleotides in defined regions on 16S rRNA and were constructed by multiple rounds of by enzymatic inverse PCR using the library construction primers in Supplementary Table 1. Each library has a diversity of greater than 10⁹, ensuring more than 99% coverage. There is overlap in the nucleotides mutated in the 11 libraries and overall they cover the entire surface of decoding centre in the A site of the ribosome.

To create a reporter of quadruplet decoding by orthogonal ribosomes, we used a previously described O-cat (UAGA146)/tRNA(UAGA) vector as a template⁹. This vector contains a variant of E. coli tRNA^{Ser2} on an Ipp promoter and rrnC transcriptional terminator. The tRNA has an altered anticodon and selector codons for serine 146 in the chloramphenicol acetyl transferase (cat) gene downstream of an orthogonal ribosome-binding site. Ser146 is an essential and conserved catalytic serine residue that ensures the fidelity of incorporation. To create O-cat (AAGA 103 AAGA146)/tRNA(UCUU) the AAGA codon was introduced at position 146 and 103 and the anticodon of the tRNA was converted to UCUU by Quikchange mutagenesis using primers CAT146AGGAf, CAT146AGGAr and CAT103AGGAf, CAT103AGGAr. O-cat reporters containing the quadruplet codons AGGA, CCCU (using primers CAT146CCCUf, CAT146CCCUr and CAT103CCCUf and CAT103CCUr) and the corresponding tRNAs (Ser2AGGAf, Ser2AGGAr, Ser2CCCUf and Ser2CCCUr) were also created by Quikchange mutagenesis. Reporters containing a single quadruplet selector codon were intermediates in the vector construction process. Vectors having the O-cat gene but lacking the tRNA were created using O-cat(UAGA146), which does not contain the tRNA cassette, as a template using Quik change primers CAT146AAGf, CAT146AGGAr, CAT103AGGAf, CAT103AGGAr, CAT146CCCUf, CAT146CCCUr, CAT103CCCUf and CAT103CCCUr that mutate the codons in O-cat.

### Selection of Orthogonal Ribosomes with Enhanced Quadruplet Decoding

To select O-ribosomes with improved quadruplet decoding, each pRSF-O-rDNA library was transformed by electroporation into GeneHog E. coli (Invitrogen) cells containing O-cat (AAGA146). Transformed cells were recovered for 1 h in SOB medium containing 2% glucose and used to inoculate 200 ml of LB-GKT (LB medium with 2% glucose, 25 μg ml⁻¹ kanamycin and 12.5 μg ml⁻¹ tetracycline). After overnight growth (37° C., 250 r.p.m., 16 h), 2 ml of the cells were pelleted by centrifugation (3,000 g), and washed three times with an equal volume of LB-KT (LB medium with 12.5 μg ml⁻¹ kanamycin and 6.25 μg ml⁻¹ tetracycline). The resuspended pellet was used to inoculate 18 ml of LB-KT, and the resulting culture incubated (37° C., 250 r.p.m. shaking, 90 min). To induce expression of plasmid encoded O-rRNA, 2 ml of the culture was added to 18 ml LB-IKT (LB medium with 1.1 mM isopropyl-D-thiogalactopyranoside (IPTG), 12.5 μg ml⁻¹ kanamycin and 6.25 μg ml⁻¹ tetracycline) and incubated for 4 h (37° C., 250 r.p.m.). Aliquots (250 ml optical density at 600 nm (OD₆₀₀)=1.5) were serial diluted and plated on LB-IKT agar (LB agar with 1 mM IPTG, 12.5 μg ml⁻¹ kanamycin and 6.25 μg ml⁻¹ tetracycline) supplemented with chloramphenicol of different concentrations (75 μg ml⁻¹, 100 μg ml⁻¹, 150 μg ml⁻¹, and 200 μg ml⁻¹ respectively) and incubated (37° C., 40 h).

### Characterization of Evolved Orthogonal Ribosomes with Enhanced Quadruplet Decoding

To separate selected pRSF-O-rDNA plasmids from the O-cat (AAGA146)/tRNA^{ser2}(UCUU) reporter plasmids, total plasmid DNA from selected clones was purified and digested with NotI restriction endonuclease, and transformed into DH10B E. coli. Individual transformants were replica plated onto kanamycin agar and tetracycline agar and plasmid separation of pRSF-O-rDNA from the reporter confirmed by restriction digest and agarose gel analysis.

To quantify the quadruplet decoding activity of selected 16S rDNA clones, the selected pRSF-O-rDNA plasmids were cotransformed with O-cat (AGGA103, AGGA146)/tRNA^{ser2}(UCCU). Cells were recovered (SOB, 2% glucose, 1 h) and used to inoculate 10 ml of LB-GKT, which was incubated (16 h, 37° C., 250 r.p.m.). We used 1 ml of the resulting culture to inoculate 9 ml of LB-KT, which was incubated (90 min, 37° C., 250 r.p.m.). We used 1 ml of the LB-KT culture to inoculate 9 ml of LB-IKT medium, which was incubated (37° C., 250 r.p.m., 4 h). Individual clones were transferred to a 96-well block and arrayed, using a 96-well pin tool, onto LB-IKT agar plates containing chloramphenicol at concentrations from 0 to 500 μg ml⁻¹. The plates were incubated (37° C., 16 h). We performed analogous experiments for other quadruplet codon-anticodon pairs.

To extract soluble cell lysates for in vitro CAT assays, 1 ml of each induced LB-IKT culture was pelleted by centrifugation at 3,000 g. The cell pellets were washed three times with 500 μl Washing Buffer (40 mM Tris-HCl, 150 mM NaCl, 1 mM EDTA, pH 7.5) and once with 500 μl lysis buffer (250 mM Tris-HCl, pH 7.8). Cells were lysed in 200 μl Lysis Buffer by five cycles of flash-freezing in dry ice/ethanol, followed by rapid thawing in a 50° C. water bath. Cell debris was removed from the lysate by centrifugation (12,000 g, 5 min) and the top 150 μl of supernatant frozen at −20° C. To assay CAT activity in the lysates, 10 μl of soluble cell extract was mixed with 2.5 μl of FAST CAT Green (deoxy) substrate (Invitrogen) and preincubated (37° C., 5 min). We added 2.5 μl of 9 mM acetyl-CoA (Sigma), and incubated (37° C., 1 h). The reaction was stopped by the addition of ice-cold ethyl acetate (200 I, vortex 20 s). The aqueous and organic phases were separated by centrifugation (12,000 g, 10 min) and the top 100 μl of the ethyl acetate layer collected. We spotted 1 μl of the collected solution onto a silica gel Thin-layer chromatography plate (Merck) for thin-layer chromatography in chloroform:methanol (85:15 vol/vol). The fluorescence of the spatially resolved substrate and product was visualized and quantified using a phosphorimager (Storm 860, Amersham Biosciences) with excitation and emission wavelengths of 450 nm and 520 nm, respectively.

### Small Scale Expression and Purification of gst-malE Fusions

E. coli (containing the appropriate plasmid combinations were pelleted (3,000 g, 10 min) from 50 ml overnight cultures, resuspended and lysed in 800 μl Novagen BugBuster Protein Extraction Reagent (supplemented with 1× protease inhibitor cocktail (Roche), 1 mM PMSF, 1 mg ml⁻¹ lysozyme (Sigma), 1 mg ml⁻¹ DNase I (Sigma)), and incubated (60 min, 25° C., 1,000 r.p.m.). The lysate was clarified by centrifugation (6 min, 25,000 g, 2° C.). GST containing proteins from the lysate were bound in batch (1 h, 4° C.) to 50 μl of glutathione sepharose beads (GE Healthcare). Beads were washed 3 times with 1 ml PBS, before elution by heating for 10 min at 80° C. in 60 μl 1× SDS gel-loading buffer. All samples were analyzed on 10% Bis-Tris gels (Invitrogen).

### Measuring the Translational Fidelity of Orthogonal Quadruplet Decoding Ribosomes

³⁵S-cysteine misincorporation: E. coli containing either pO-gst-malE and pSC101*-O-ribosome, pO-gst-malE and pSC101*-ribo-X, pO-gst-malE and pSC101*-riboQ, or pgst-malE were resuspended in LB media (supplemented with ³⁵S-cysteine (1,000 Ci mmol⁻¹) to a final concentration of 3 nM, 750 μM methionine, 25 μg ml⁻¹ ampicillin and 12.5 μg ml⁻¹ kanamycin) to an OD600 of 0.1, and cells were incubated (3.5 h, 37° C., 250 r.p.m.). 10 ml of the resulting culture was pelleted (5,000 g, 5 min), washed twice (1 ml PBS per wash), resuspended in 1 ml lysis buffer containing 1% Triton-X, incubated (30 min, 37° C., 1,000 r.p.m.) and lysed on ice by pipetting up and down. The clarified cell extract was bound to 100 μl of glutathione sepharose beads (1 h, 4° C.) and the beads were pelleted (5,000 g, 10 s) and washed twice in 1 ml PBS. The beads were added to 10 ml polypropylene column (Biorad) and washed (30 ml of PBS; 10 ml 0.5 M NaCl, 0.5× PBS; 30 ml PBS) before elution in 1 ml of PBS supplemented with 10 mM glutathione. Purified GST-MBP was digested with 12.5 units of thrombin for 1 h, to yield a GST fragment and an MalE fragment. The reaction was precipitated with 15% trichloroacetic acid and loaded onto an SDS-PAGE gel to resolve the GST, MBP and thrombin, and stained with InstantBlue (Expedeon). The ³⁵S activity in the GST and MBP protein bands were quantified by densitometry, using a Storm Phosphorimager (Molecular Dynamics) and ImageQuant (GE Healthcare). The error frequency per codon for each ribosome examined was determined as follows: GST contains four cysteine codons, so the number of counts per second (c.p.s.) resulting from GST divided by four gives A, the cps per quantitative incorporation of cysteine. MBP contains no cysteine codons, but misincorporation at noncysteine codons gives B c.p.s. Because GST and MBP are present in equimolar amounts, (A/B 410, where 410 is the number of amino acids in the MBP containing thrombin cleavage fragment, gives the number of amino acids translated for one cysteine misincorporation C. Assuming the misincorporation frequency for all 20 amino acids is the same as that for cysteine the number of codons translated per misincorporation is C/20, and the error frequency per codon is given by (C/20)⁻¹.

Dual luciferase assays: The previously characterized pO-DLR contains a genetic fusion between a 5′ Renilla luciferase (R-luc) and a 3′ firefly luciferase (F-luc) on an orthogonal ribosome binding site⁹. pO-DLR, and its K529 codon variants, were transformed into E. coli cells with pSC101*-O-ribosome or pSC101*-ribo-Q1. Where indicated an additional E. coli Ser2A tRNA with a mutated anticodon, as specified in individual experiments, was supplied on plasmid p15A-tRNA-Ser2A. In this case 25 μg ml⁻¹ tetracyclin was added to all culture media to maintain the additional plasmid. In experiments that used a suppressor tRNA recognizing AGGA codons a natural AGG codon, that is followed by a codon starting with an A, was removed from the linker region of pO-DLR by QuikChange using primers DLR952AAGxf and DLR953AGGxr.

Individual colonies were incubated (37° C., 250 r.p.m., 36 h) in 2 ml LB supplemented with ampicillin (50 μg ml⁻¹) and kanamycin (25 μg ml⁻¹), pelleted (5,000 g, 5 min), washed with ice cold Millipore water and resuspended in 300 μl (1 mg ml⁻¹ lysozyme, 1 mg ml⁻¹ DNase I, 10 mM Tris (pH 8.0), 1 mM EDTA). Cells were incubated on ice for 20 min, frozen on dry ice, and thawed on ice. 10 μl samples of this extract were assayed for firefly (F-luc) and Renilla (R-luc) luciferase activity using the Dual-Luciferase Reporter Assay System (Promega). Each ribosome reporter combination was assayed from four independent cultures using an Orion microplate luminometer (Berthold Detection Systems) and the data analyzed as previously described. The error reported is the standard deviation.

### Mass Spectrometric Characterization of p-azido-L-phenylalanine (2) Incorporation by Ribo-Q1

E. coli DH10B containing p-O-gst-malE(Y252AGGA), pSC101*Ribo-Q1 and pDULE-AzPheRS*tRNA_{UCCU} were used to produce protein for mass spectrometry. Protein was expressed in the presence of 2.5 mM 2 and purified on glutathione. The purified proteins were resolved by SDS-PAGE, stained with Instant Blue (Expedeon) and the band containing full length GST-MBP was excised for analysis by LC/MS/MS (NextGen Sciences). The samples were reduced with DTT at 60° C. and alkylated with iodoacetamide after cooling to room temperature. The samples were then digested with trypsin (37° C., 4 h), and the reaction was stopped by the addition of Formic acid. The samples were analyzed by nano LC/MS/MS on a ThermoFisher LTQ Orbitrap XL. 30 μl of hydrolysate was loaded onto a 5 mm 75 μm ID C12 (Jupiter Proteo, Phenomenex) vented column at a flow-rate of 10 μl min⁻¹. Gradient elution was over a 15 cm 75 μm ID C12 column at 300 nl min⁻¹ with a 1 hour gradient. The mass spectrometer was operated in data-dependent mode, and ions were selected for MS/MS. The Orbitrap MS scan was performed at 60,000 FWHM resolution. MS/MS data was searched using Mascot (www.matrixscience.com).

### Evolution of a Quadruplet Decoding MjAzPheRS

pBK MjAzPheRS-7²⁴ (a kanamycin resistant plasmid, which contains MjAzPheRS-7 on a GInRS promoter and terminator) was used as a template to create a library in the region of MjAzPheRS that recognizes the anticodon. Codons for residues Y230, C231, P232, F261, H283 and D286 were randomized to NNK in two rounds of enzymatic inverse PCR, generating a library of 10⁸ mutant clones. pREP JY(UCCU) was created by changing the anticodon of MjtRNA_{CUA} in pREP YC-JYCUA³² from CUCUAAA to CUUCCUAA by QuikChange mutagenesis (Stratagene) and changing the amber codon in the chloramphenicol acetyltransferase gene to AGGA. E. coli DH10B harbouring this plasmid were transformed with the mutant library and grown in LB-KT (LB medium supplemented with 25 μg ml⁻¹ kanamycin and 12.5 μg ml⁻¹ tetracycline) supplemented with 1 mM 2. 10⁹ cells were plated on LB-KT plates containing 1 mM 2 and concentrations of chloramphenicol ranging from 50 to 250 μg ml⁻¹. After incubation (36 h, 37° C.) individual clones were tested for 2 dependent growth on LB-KT plates with 0-250 μg ml⁻¹ chloramphenicol with and without 1 mM 2. The plasmid DNA from clones showing amino acid dependent growth was isolated and digested with HindIII to eliminate pREP JY(UCCU). After transformation and reisolation of the kanamycin resistant plasmid the DNA was sequenced.

To select quadruplet decoding pairs that incorporate other amino acids, the procedure above was repeated using the relevant starting template and unnatural amino acid.

### Investigating the Mutual Orthogonality of MbPyIRS/MbtRNA_{CUA} and MjTyrRS/MjtRNA_{CUA}

To test the ability of MbPyIRS to aminoacylate MjtRNA_{CUA} E. coli DH10B were transformed with a pBK MbPyIRS encoding MbPyIRS under the control of a GInRS promoter and terminator and pMyo4TAG-His₆, expressing sperm whale myoglobin with an amber codon at position 4 and MjtRNA_{CUA}. The cells were grown overnight at 37° C. in LB-KT. Fresh LB-KT (50 ml) supplemented with 10 mM N6-[(tert.-butyloxy)carbonyl]-L-lysine (BocLys, 3) was inoculated 1:50 with overnight culture. After 3 h at 37° C. protein expression was induced by addition of 0.2% arabinose. After a further 3 h cells were harvested and washed with PBS. Proteins were extracted by shaking at 25° C. in 1 ml Ni-wash buffer (10 mM Tris/CI, 20 mM imidiazole, 200 mM NaCl pH 8.0) supplemented with protease inhibitor cocktail (Roche), 1 mM PMSF, and approx. 1 mg ml⁻¹ lysozyme and 0.1 mg ml⁻¹ DNAse I. The extract was clarified by centrifugation (5 min, 25000 g, 4° C.), supplemented 50 μl Ni²⁺-NTA beads and incubated with agitation for 1 h at 4° C. Beads were washed in batch three times with 1 ml Ni-wash buffer and eluted in 100 μl sample buffer supplemented with 200 mM imidazole. To test the aminoacylation activity between the cognate pairs or between MjTyrRS and MbtRNA_{CUA} analogous experiments were carried out as above using the relevant plasmids (pBK MjTyrRS or pBK MbPyIRS and pMyo4TAG-His₆ or pMyo4TAG-His₆-PyIT) and unnatural amino acids (3 or none). Proteins were analysed by 4-12% SDS-PAGE and stained with Instant Blue.

### Characterization of the Quadruplet Suppressing AzPheRS*

Expression and purification of myoglobin from pMyo4TAG-His₆ or pMyo4AGGA-His₆ was carried out as above using the relevant pBK plasmids and 2.5 mM 2. Proteins were analysed by 4-12% SDS-PAGE.

### Characterization of Myo4AzPhe produced with AzPheRS* from pMyo4AGGA-His₆ by ESI Mass Spectrometry

Myoglobin was expressed in E. coli DH10B using plasmids pBK AzPheRS* and pMyo4AGGA-His₆ essentially as described above but at 1 I scale. The protein was extracted by shaking at 25° C. in 30 ml Ni-wash buffer supplemented with protease inhibitor cocktail (Roche), 1 mM PMSF, 1 mg ml⁻¹ lysozyme and 0.1 mg ml⁻¹ DNAse I. The extract was clarified by centrifugation (15 min, 38000 g, 4° C.), supplemented 0.3 ml Ni²⁺-NTA beads and incubated with agitation for 1 h at 4° C. Beads were poured into a column and washed with 40 ml of Ni-wash buffer. Bound protein was eluted in 0.5 ml fractions of the same buffer containing 200 mM imidazole and immediately rebuffered to 10 mM ammonium carbonate pH 7.5 by dialysis. 50 μl of the sample was mixed 1:1 with 1% formic acid in 50% methanol and total mass determined on an LCT time-of-flight mass spectrometer with electrospray ionization (Micromass). The sample was injected at 10 μl min⁻¹ and calibration performed in positive ion mode using horse heart myoglobin. 50 scans were averaged and molecular masses obtained by deconvoluting multiply charged protein mass spectra using MassLynx version 4.1 (Micromass). The theoretical mass of the wild-type myoglobin was calculated using Protparam (http://us.expasy.org/tools/protparam.html), and the theoretical mass for 2 adjusted manually.

### MS/MS Analysis of GST-MBP 234AzPhe 239CAK

E. coli DH10B were transformed with pDULE AzPheRS*/tRNA_{UCCU} and pCDF PyIST and grown to logarithmic phase in LB-ST (25 μg ml⁻¹ spectinomycin and 12.5 μg ml⁻¹ tetracycline). Electrocompetent cells were prepared and transformed with a plasmid for the constitutive expression of an orthogonal ribosome (pSC101* Ribo-Q) and p-O-gst(234AGGA 239TAG)malE. The recovery of the transformation was used to inoculate LB-AKST (LB medium containing 50 μg ml⁻¹ ampicillin, 12.5 μg ml⁻¹ kanamycin, 25 μg ml⁻¹ spectinomycin and 12.5 μg ml⁻¹ tetracycline). The culture was grown to saturation at 37° C. and used to inoculate the main culture 1:50. Cells were grown overnight at 37° C., harvested by centrifugation and stored at −20° C. The GST-MBP protein was expressed at a scale of 100 ml using 2.5 mM of each AzPhe (2) and CAK (4). Proteins were extracted and purified as above. After washing the beads with PBS the protein was eluted by heating in 100 μl 1× sample buffer containing 50 mM β-mercaptoethanol to 80° C. for 5 min. The protein sample was analysed by 4-12% SDS-PAGE and stained with Instant Blue. The band containing full-length GST-MBP was excised and submitted for LC/MS/MS analysis (by NextGen Sciences).

### Cyclization of GST-COM-His₆ 1AzPhe 149CAK

E. coli DH10B were transformed sequentially with four plasmids as described above using expression plasmids p-O-gst-CaM-His₆ 1 AGGA 149UAG or p-O-gst-COM-His₆ 1AGGA 40UAG. The protein was expressed at 0.5 L scale as described above using 5 mM 2 and 2.5 mM 4. The cells were extracted and GST-CaM-His₆ purified as described for myoglobin-His₆ and dialysed against 50 mM Na₂HPO₄ pH 8.3. To perform the cyclization reaction, 160 μl of protein sample was mixed with 40 μl of a fresh solution of 5 mM ascorbic acid, 5 mM CuSO4 and 10 mM bathophenanthroline. The reaction was incubated at 4° C. and analysed by 4-12% SDS-PAGE.

To analyze the cyclization product by mass spectrometry we introduced additional tryptic cleavage sites around the incorporation sites of unnatural amino acids to facilitate subsequent analysis. Therefore, the point mutations Q4K and M146K (numbering relative to the AGGA codon in p-O-gst-CaM-His₆ 1AGGA 149UAG) and a G₃K linker directly following the TAG codon were introduced by QuikChange. The protein was expressed, purified and cyclized as above with very similar yields. The cyclized protein was subsequently excised from an SDS-PAGE gel and submitted for mass spectrometric analysis (NextGen Sciences, Ann Arbor, USA).

### References

1. Xie, J. & Schultz, P. G. A chemical toolkit for proteins--an expanded genetic code. Nat Rev Mol Cell Biol 7, 775-82 (2006).
2. Steer, B. A. & Schimmel, P. Major anticodon-binding region missing from an archaebacterial tRNA synthetase. J Biol Chem 274, 35601-6 (1999).
3. Chin, J. W., Martin, A. B., King, D. S., Wang, L. & Schultz, P. G. Addition of a photocrosslinking amino acid to the genetic code of Escherichiacoli. Proc Natl Acad Sci U S A 99, 11020-4 (2002).
4. Srinivasan, G., James, C. M. & Krzycki, J. A. Pyrrolysine encoded by UAG in Archaea: charging of a UAG-decoding specialized tRNA. Science 296, 1459-62 (2002).
5. Polycarpo, C. et al. An aminoacyl-tRNA synthetase that specifically activates pyrrolysine. Proc Natl Acad Sci USA 101, 12450-4 (2004).
6. Neumann, H., Peak-Chew, S. Y. & Chin, J. W. Genetically encoding N(epsilon)-acetyllysine in recombinant proteins. Nat Chem Biol 4, 232-4 (2008).
7. Nguyen, D. P. et al. Genetic encoding and labeling of aliphatic azides and alkynes in recombinant proteins via a pyrrolysyl-tRNA Synthetase/tRNA(CUA) pair and click chemistry. J Am Chem Soc 131, 8720-1 (2009).
8. Rackham, O. & Chin, J. W. A network of orthogonal ribosome x mRNA pairs. Nat Chem Biol 1, 159-66 (2005).
9. Wang, K., Neumann, H., Peak-Chew, S. Y. & Chin, J. W. Evolved orthogonal ribosomes enhance the efficiency of synthetic genetic code expansion. Nat Biotechnol 25, 770-7 (2007).
10. Rostovtsev, V. V., Green, L. G., Fokin, V. V. & Sharpless, K. B. A stepwise huisgen cycloaddition process: copper(I)-catalyzed regioselective “ligation” of azides and terminal alkynes. Angew Chem Int Ed Engl 41, 2596-9 (2002).
11. Hohsaka, T. & Sisido, M. Incorporation of non-natural amino acids into proteins. Curr Opin Chem Biol 6, 809-15 (2002).
12. Ohtsuki, T., Manabe, T. & Sisido, M. Multiple incorporation of non-natural amino acids into a single protein using tRNAs with non-standard structures. FEBS Lett 579, 6769-74 (2005).
13. Murakami, H., Hohsaka, T., Ashizuka, Y. & Sisido, M. Site-directed incorporation of p-nitrophenylalanine into streptavidin and site-to-site photinduced electron transfer from a pyrenyl group to a nitrophenyl group on the protein framework. Journal of the American Chemical Society 120, 7520-7529 (1998).
14. Rodriguez, E. A., Lester, H. A. & Dougherty, D. A. In vivo incorporation of multiple unnatural amino acids through nonsense and frameshift suppression. Proc Natl Acad Sci U S A 103, 8650-5 (2006).
15. Monahan, S. L., Lester, H. A. & Dougherty, D. A. Site-specific incorporation of unnatural amino acids into receptors expressed in mammalian cells. Chemistry and Biology 10, 573-580 (2003).
16. Anderson, J. C. et al. An expanded genetic code with a functional quadruplet codon. Proc Natl Acad Sci U S A 101, 7566-71 (2004).
17. Atkins, J. F. & Bjork, G. R. A gripping tale of ribosomal frameshifting: extragenic suppressors of frameshift mutations spotlight P-site realignment. Microbiol Mol Biol Rev 73, 178-210 (2009).
18. Stahl, G., McCarty, G. P. & Farabaugh, P. J. Ribosome structure: revisiting the connection between translational accuracy and unconventional decoding. Trends Biochem Sci 27, 178-83 (2002).
19. Selmer, M. et al. Structure of the 705 ribosome complexed with mRNA and tRNA. Science 313, 1935-42 (2006).
20. Magliery, T. J., Anderson, J. C. & Schultz, P. G. Expanding the genetic code: selection of efficient suppressors of four-base codons and identification of “shifty” four-base codons with a library approach in Escherichia coli. J Mol Biol 307, 755-69 (2001).
21. Khazaie, K., Buchanan, J. H. & Rosenberger, R. F. The accuracy of Q beta RNA translation. 1. Errors during the synthesis of Q beta proteins by intact Escherichia coli cells. Eur J Biochem 144, 485-9 (1984).
22. Laughrea, M., Latulippe, J., Filion, A. M. & Boulet, L. Mistranslation in twelve Escherichia coli ribosomal proteins. Cysteine misincorporation at neutral amino acid residues other than tryptophan. Eur J Biochem 169, 59-64 (1987).
23. Kramer, E. B. & Farabaugh, P. J. The frequency of translational misreading errors in E. coli is largely determined by tRNA competition. Rna 13, 87-96 (2007).
24. Chin, J. W. et al. Addition of p-azido-L-phenylalanine to the genetic code of Escherichia coli. J Am Chem Soc 124, 9026-7 (2002).
25. Mukai, T. et al. Adding l-lysine derivatives to the genetic code of mammalian cells with engineered pyrrolysyl-tRNA synthetases. Biochem Biophys Res Commun 371, 818-22 (2008).
26. Camarero, J. A., Pavel, J. & Muir, T. W. Chemical Synthesis of a Circular Protein Domain: Evidence for Folding-Assisted Cyclization. Angewandte Chemie—International Edition 37, 347-349 (1998).
27. Scott, C. P., Abel-Santos, E., Wall, M., Wahnon, D. C. & Benkovic, S. J. Production of cyclic peptides and proteins in vivo. Proc Natl Acad Sci U S A 96, 13638-43 (1999).
28. Li, P. & Roller, P. P. Cyclization strategies in peptide derived drug design. Curr Top Med Chem 2, 325-41 (2002).
29. Walensky, L. D. et al. Activation of apoptosis in vivo by a hydrocarbon-stapled BH3 helix. Science 305, 1466-70 (2004).
30. Trauger, J. W., Kohli, R. M., Mootz, H. D., Marahiel, M. A. & Walsh, C. T. Peptide cyclization catalysed by the thioesterase domain of tyrocidine synthetase. Nature 407, 215-8 (2000).
31. Stemmer, W. P. & Morris, S. K. Enzymatic inverse PCR: a restriction site independent, single-fragment method for high-efficiency, site-directed mutagenesis. Biotechniques 13, 214-20 (1992).
32. Santoro, S. W., Wang, L., Herberich, B., King, D. S. & Schultz, P. G. An efficient system for the evolution of aminoacyl-tRNA synthetase specificity. Nat Biotechnol 20, 1044-8 (2002).
33. Rice, J. B., Libby, R. T. & Reeve, J. N. Mistranslation of the mRNA encoding bacteriophage T7 0.3 protein. J Biol Chem 259, 6505-10 (1984).
34. Hao, B. et al. A new UAG-encoded residue in the structure of a methanogen methyltransferase. Science 296, 1462-6 (2002).
35. Kobayashi, T. et al. Structural basis for orthogonal tRNA specificities of tyrosyl-tRNA synthetases for genetic code expansion. Nat Struct Biol 10, 425-32 (2003).

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A 16S rRNA comprising a mutation at A1196.

2. A 16S rRNA according to claim 1 wherein said mutation is A1196G.

3. A 16S rRNA according to claim 1 further comprising a mutation at C1195 and/or A1197.

4. A 16S rRNA according to claim 1 comprising mutations that are
(i) C1195A and A1196G; or
(ii) C1195T, A1196G and A1197G; or
(iii) A1196G and A1197G.

5. A 16S rRNA according to claim 1, further comprising mutations at A531 G and U534A.

6. A ribosome capable of translating a quadruplet codon, said ribosome comprising a 16S rRNA comprising a mutation at A1196.

7-9. (canceled)

10. A 16S rRNA according to claim 2 further comprising a mutation at C1195 and/or A1197.

11. A ribosome according to claim 6 wherein said mutation is A1196G.

12. A ribosome according to claim 6, wherein the 16S rRNA further comprises a mutation at C1195 and/or A1197.

13. A ribosome according to claim 11, wherein the 16S rRNA further comprises a mutation at C1195 and/or A1197.

14. A ribosome according to claim 6, wherein the 16S rRNA comprises mutations that are
(i) C1195A and A1196G; or
(ii) C1195T, A1196G and A1197G; or
(iii) A1196G and A1197G.

15. A ribosome according to claim 6, wherein the 16S rRNA further comprises mutations at A531 G and U534A.

16. A method for translating an mRNA comprising
contacting an mRNA, said mRNA comprising at least one quadruplet codon, with a ribosome capable of translating a quadruplet codon, said ribosome comprising a 16S rRNA comprising a mutation at A1196.

17. A method according to claim 16, wherein said mutation is A1196G.

18. A method according to claim 16, wherein the 16S rRNA further comprises a mutation at C1195 and/or A1197.

19. A method according to claim 17, wherein the 16S rRNA further comprises a mutation at C1195 and/or A1197.

20. A method according to claim 16, wherein the 16S rRNA comprises mutations that are
(i) C1195A and A1196G; or
(ii) C1195T, A1196G and A1197G; or
(iii) A1196G and A1197G.

21. A method according to claim 16, wherein the 16S rRNA further comprises mutations at A531 G and U534A.
